(19) United States
(12) Patent Application Publication
(10) Pub. No.: US 2025/0135442 A1
(43) Pub. Date: May 1, 2025

(54) COPPER-LOADED MORDENITE ZEOLITE (Cu-MOR) CATALYST AND PROCESS FOR DIRECT METHANE OXIDATION TO METHANOL

(71) Applicant: KING FAHD UNIVERSITY OF PETROLEUM AND MINERALS, Dhahran (SA)

(72) Inventors: Khalid ALHOOSHANI, Dhahran (SA); Ijaz HUSSAIN, Dhahran (SA)

(73) Assignee: KING FAHD UNIVERSITY OF PETROLEUM AND MINERALS, Dhahran (SA)

(21) Appl. No.: 18/497,454

(22) Filed: Oct. 30, 2023

(51) Int. Cl. B01J 29/24; B01J 37/04; B01J 37/08; C07C 29/50; C07C 29/74

(52) U.S. Cl. CPC B01J 29/24; B01J 37/04; B01J 37/08; C07C 29/50; C07C 29/74; B01J 2229/186

(57) ABSTRACT

A method for direct methane (CH₄) oxidation (DMTM) to methanol (CH₃OH) includes passing an oxygen-containing feed gas stream into a reactor containing a copper-loaded mordenite zeolite (Cu-MOR) catalyst particles such that the oxygen-containing feed gas stream is in contact with the Cu-MOR catalyst particles, at a temperature of 100 to 500° C., to form an oxidized Cu-MOR catalyst. The method further includes displacing oxygen in the reactor by nitrogen purging, and further passing a CH₄-containing feed gas stream through the reactor in contact with the oxidized Cu-MOR catalyst at a temperature of 50 to 200° C., thereby converting at least a portion of the CH₄ to CH₃OH. The method further includes regenerating the Cu-MOR catalyst particles to form a regenerated Cu-MOR catalyst. The CH₃OH is adsorbed on surfaces and pores of the regenerated Cu-MOR catalyst.

### STATEMENT REGARDING PRIOR DISCLOSURE BY THE INVENTORS

Aspects of this technology are described in I. Hussain, S. Ganiyu, H. Alasiri, and K. Alhooshani, “Highly dispersed Cu-anchored nanoparticles based mordenite zeolite catalyst (Cu-MOR): Influence of the different preparation methods for direct methane oxidation (DMTM) to methanol” published in Journal of the Energy Institute, Volume 109, 101269, which is incorporated herein by reference in its entirety.

### STATEMENT OF ACKNOWLEDGEMENT

This research was supported by the Center for Refining and Advanced Chemicals, Research Institute at King Fahd University of Petroleum and Minerals (KFUPM), Dhahran, Saudi Arabia, under the Project INRC2216.

### BACKGROUND

### Technical Field

The present disclosure is directed to a zeolite catalyst, and particularly to a copper-loaded mordenite zeolite (Cu-MOR) catalyst for direct oxidation of methane to methanol.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Since the beginning of the industrial revolution, human activities have increased, leading to mounting emissions of greenhouse gases and other environmental issues. Among other gases, methane harms the atmosphere; since it absorbs short- and long-wave radiation and has a radiative force of 0.61 W/m². In addition to minor emissions from oceans, termites, sedimentary waterlogging, and inland groundwater, the most common places where methane is released are wetland areas, agricultural areas (including waste, ruminants, and rice fields), the combustion of detritus, industrial emissions, and biofuels. Due to radical hydroxyl oxidation, soil microorganism consumption, and contact with chlorine atoms, the lifetime of CH₄ is approximately nine years. Between 2010 and 2019, the global mean methane level rose by 4%, going from 1798 to 1866 ppb, with an annual growth rate ranging from 5 to 13 ppb/year in the global atmosphere. Despite methane's limited direct use as a fuel and challenges related to its transportation and storage, there is a growing interest in technological advancements to convert it into liquid fuels and for various industrial applications, especially on a regional and on-site basis. However, methane conversion is highly challenging due to its unique molecular composition. It can only be activated under extremely restrictive conditions due to its regular tetrahedral configuration, a significant strength of the C—H bond (104 kcal/mol), minimal electron affinity, and negligible polarizability. After activating the first C—H bond in methane, reaction intermediates and derivatives become much more reactive and transformable.

Research and industrial organizations have increasingly focused on effective and controllable methane conversion due to the inherent challenges in managing the reaction process. The primary commercial methods for methane utilization include but are not limited to the Fischer-Tropsch process, methanol synthesis, immediate transformation by dehydro-aromatization reaction over metal-zeolite, and oxidative coupling reaction of ethylene over metal oxide. Academic and industrial sectors are paying close attention to methane's direct and selective oxidation to yield valuable chemicals. However, this process is complicated by the product's higher reactivity compared to methane, which can lead to over-oxidation, making it more challenging. One approach to mitigate over-oxidation involves synthesizing intermediates that can help stabilize the end product.

Selective oxidation of methane to methanol may be achieved by using pentasil zeolites such as Zeolite Socony Mobil-5 (ZSM-5) and mordenite (MOR). These materials stabilize binuclear oxygen-bridging centers of Fe and Cu, which are structurally similar to the active metal centers found in methane mono-oxygenase (MMO) in methanotrophic bacteria. MMO catalyzes the conversion of methane into methanol. Active sites like Fe-ZSM-5, Cu-ZSM-5, Co-ZSM-5, and Cu-MOR facilitate the activation of the C—H linkage in methane and stabilize reaction intermediates during the oxidation process. Oxygen activation leads to the formation of active metal-oxygen species, and their reaction with CH₄ forms a chemisorbed intermediate. The utilization of a wet gas stream under reaction conditions or solvent extraction as part of the reactive treatment results in the desorption of the product in the form of methanol. In Cu-based catalysts, such as Cu-CHA, Cu-MOR, and Cu-MFI, [Cu₂O]²⁺, active sites were discovered during the direct gas-phase conversion of methane to methanol (P. Vanelderen, B. E. Snyder, M. L. Tsai, R. G. Hadt, J. Vancauwenbergh, O. Coussens, R. A. Schoonheydt, B. F. Sels, E. I. Solomon, Spectroscopic definition of the copper active sites in mordenite: selective methane oxidation, J. Am. Chem. Soc. 137 (19) (2015) 6383-6392; and B. E. Snyder, P. Vanelderen, M. L. Bols, S. D. Hallaert, L. H. B. Ottger, L. Ungur, K. Pierloot, The active site of low-temperature methane hydroxylation in iron-containing zeolites, Nature 536 (7616) (2016) 317-321).

Methane can undergo hydroxylation to produce methanol in various configurations by α-O, which is generated by O₂ or N₂O. Unlike the α-O site in Fe-zeolites, which can react at room temperature, these [Cu₂O]²⁺ sites can be generated with O₂ or N₂O, making them less reactive with methane. By adjusting the availability of O₂ with delayed activation time, the structure-activity relationship between methane to methanol over Cu-CHA catalyst can be described (H. M. Rhoda, D. Plessers, A. J. Heyer, M. L. Bols, R. A. Schoonheydt, B. F. Sels, E. I. Solomon, Spectroscopic definition of a highly reactive site in Cu-CHA for selective methane oxidation: tuning a mono-μ-oxo dicopper (II) active site for reactivity, J. Am. Chem. Soc. 143 (19) (2021) 7531-7540). In terms of energy efficiency, employing a liquid-phase solvent for moderate methane oxidation offers significant advantages. At low temperatures, a strong oxidant could produce more liquid methanol (V. L. Sushkevich, D. Palagin, M. Ranocchiari, J. A. Van Bokhoven, Selectiveanaerobic oxidation of methane enables direct synthesis of methanol, Science 356 (6337) (2017) 523-527; and L. Yang, J. Huang, R. Ma, R. You, H. Zeng, Z. Rui, Metal-organic framework-derived IrO₂/CuO catalyst for selective oxidation of methane to methanol, ACS Energy Lett. 4 (12) (2019) 2945-2951). Under appropriate reaction conditions and with various oxidants, including O₂, N₂O, and H₂O₂, Cu active sites can improve methanol selectivity during methane oxidation. For commercial applications, using O₂ as an oxidant is inevitable due to the economic impracticality of H₂O₂ (A. I. Olivos-Suarez, ‘A. Sz'ecs’ enyi, E. J. Hensen, J. Ruiz-Martinez, E. A. Pidko, J. I. Gascon, Strategies for the direct catalytic valorization of methane using heterogeneous catalysis: challenges and opportunities, ACS Catal. 6 (5) (2016), 2965-81). It has been found that Fe, Co, and Cu-loaded zeolites activated in O₂ or N₂O can react with methane at low temperatures (e.g., 200° C.) and then produce methanol when exposed to water or organic solvents (acetonitrile, ethanol, tetrahydrofuran) (E. V. Starokon, M. V. Parfenov, S. S. Arzumanov, L. V. Pirutko, A. G. Stepanov, G. I. Panov, Oxidation of methane to methanol on the surface of FeZSM-5 zeolite, J. Catal. 300 (2013) 47-54; and M. V. Parfenov, E. V. Starokon, L. V. Pirutko, G. I. Panov, Quasicatalytic and catalytic oxidation of methane to methanol by nitrous oxide over FeZSM-5 zeolite, J. Catal. 318 (2014) 14-21; and P. Tomkins, A. Mansouri, S. E. Bozbag, F. Krumeich, M. B. Park, E. M. Alayon, M. Ranocchiari, J. A. van Bokhoven, Isothermal cyclic conversion of methane into methanol over copper-exchanged zeolite at low temperature, Angew. Chem. 128 (18) (2016) 5557-5561).

Although a few catalytic methods have been described in the past for methane production, the methanol yield was limited and economically impractical. Hence, new and efficient strategies for minimizing over-oxidation to increase methanol selectivity are required. In view of the foregoing, one object of the present disclosure is to provide methods for producing methanol from methane that overcome the limitations of the art.

### SUMMARY

In an exemplary embodiment, a method for direct methane (CH₄) oxidation (DMTM) to methanol (CH₃OH), is described. The method includes introducing an oxygen-containing feed gas stream into a reactor containing a copper-loaded mordenite zeolite (Cu-MOR) catalyst containing Cu-MOR catalyst particles having a porous structure, and an average particle size of 200 to 400 micrometers (μm). In some embodiments, the Cu-MOR catalyst is at least one selected from the group consisting of a Cu-MOR (Cu-MOR-WI) catalyst made by wetness impregnation, and a Cu MOR (Cu-MOR-IWI) catalyst made by incipient wet impregnation. The method includes passing the oxygen-containing feed gas stream through the reactor in contact with the Cu-MOR catalyst particles at a temperature of 100 to 500° C. to form an oxidized Cu-MOR catalyst. The method further includes terminating the introducing the oxygen-containing feed gas stream, and introducing and passing a CH₄-containing feed gas stream through the reactor in contact with the oxidized Cu-MOR catalyst at a temperature of 50 to 200° C. thereby converting at least a portion of the CH₄ to CH₃OH and regenerating the Cu-MOR catalyst particles to form a regenerated Cu-MOR catalyst, and producing a residue gas stream leaving the reactor. In some embodiments, the CH₃OH is adsorbed on surfaces and pores of the regenerated Cu-MOR catalyst. The method further includes terminating the introducing the CH₄-containing feed gas stream and cooling the reactor, and separating and collecting the CH₃OH.

In some embodiments, the CH₄ is present in the CH₄-containing feed gas stream at a concentration of 5 to 50 vol. % based on a total volume of the CH₄-containing feed gas stream.

In some embodiments, the CH₄-containing feed gas stream further comprises an inert gas selected from the group consisting of nitrogen, argon, and helium. In some embodiments, a volume ratio of the CH₄ to the inert gas present in the CH₄-containing feed gas stream is in a range of 1:1 to 1:20.

In some embodiments, the CH₄-containing feed gas stream further comprises nitrogen. In some embodiments, the residue gas stream leaving the reactor comprises methane, nitrogen, formaldehyde, carbon monoxide, carbon dioxide, and nitrogen oxides (NOₓ).

In some embodiments, the reactor is at least one selected from the group consisting of a fixed-bed reactor, a trickle-bed reactor, a moving bed reactor, a rotating bed reactor, a fluidized bed reactor, and a slurry reactor.

In some embodiments, the reactor is a fixed-bed reactor in the form of a cylindrical reactor. The fixed-bed reactor includes a top portion; a cylindrical body portion; a bottom portion; a housing having an open top and open bottom supportably maintained with the cylindrical body portion. The Cu-MOR catalyst is supportably retained within the housing, permitting fluid flow therethrough. The fixed-bed reactor further includes at least one propeller agitator is disposed in the bottom portion of the reactor. In some embodiments, the bottom portion is cone-shaped or pyramidal. In some embodiments, a plurality of recirculation tubes fluidly connects the bottom portion of the cylindrical reactor with the cylindrical body portion of the cylindrical reactor.

In some embodiments, the method has a methanol production yield of about 10 to 30 μmol of methanol per gram of the Cu-MOR catalyst in the first 5 to 20 minutes, or even more preferably in the first 10 minutes of contacting.

In an exemplary embodiment, a method of preparing the preparing the Cu-MOR-WI is described. The method includes mixing an aluminate salt and a silica material in an alkaline solution to form a first mixture; heating the first mixture at a temperature of about 170° C. under pressure to form a crude product suspended in the first mixture; removing the crude product from the first mixture, washing and calcining at a temperature of about 550° C. to form mordenite zeolite (MOR) having a porous structure; mixing the MOR with a copper salt solution, and heating thereby depositing copper ions on surfaces and pores of the MOR; calcining the MOR comprising the copper ions at a temperature of about 550° C. to form an ion exchange Cu-MOR (Cu-MOR-IE) catalyst; mixing the Cu-MOR-IE catalyst and the copper salt solution to form a first modified Cu-MOR-IE catalyst in the form of particles suspended in the copper salt solution; and remove the first modified Cu-MOR-IE catalyst particles from the copper salt solution, and calcining at a temperature of about 550° C. to form the Cu-MOR-WI catalyst. In some embodiments, the Cu-MOR-IE catalyst has a copper content of about 2.5 wt. % based on a total weight of the Cu-MOR-IE catalyst as determined by energy-dispersive X-ray spectroscopy (EDX). In some embodiments, the Cu-MOR-IE catalyst has a methanol production yield of about 12.4 μmol of methanol per gram of the Cu-MOR-IE catalyst in the first 5 to 20 minutes, or even more preferably in the first 10 minutes of contacting.

In some embodiments, the aluminate salt is at least one selected from the group consisting of sodium aluminate and potassium aluminate.

In some embodiments, the copper salt solution comprises a copper salt selected from the group consisting of copper sulfate, copper nitrate, copper chloride, copper acetate, copper carbonate, copper phosphate, and/or a hydrate thereof.

In some embodiments, the Cu-MOR-WI catalyst comprises CuO nanoparticles having an average particle size of 7 nm.

In some embodiments, the Cu-MOR-WI catalyst has a copper content of about 3.7 wt. % based on a total weight of the Cu-MOR-WI catalyst as determined by EDX.

In some embodiments, the Cu-MOR-WI catalyst has a methanol production yield of about 26.5 μmol of methanol per gram of the Cu-MOR-WI catalyst in the first 10 minutes of contacting.

In some embodiments, the Cu-MOR-WI catalyst comprises particles having a specific surface area in a range of 290 to 300 square meter per gram (m²/g).

In some embodiments, the Cu-MOR-WI catalyst comprises particles having a mesopore volume in a range of 0.1 to 0.15 cubic centimeter per gram (cm³/g), and a micropore volume in a range of 0.15 to 0.17 cm³/g.

In some embodiments, the method further includes applying the copper salt solution on surfaces and pores of the Cu-MOR-IE catalyst to form a second Cu-MOR-IE catalyst in the form of wetted particles; and calcining the second Cu-MOR-IE catalyst at a temperature of about 550° C. to form the Cu-MOR-IWI catalyst comprising CuO nanoparticles having an average particle size of 9 nm.

In some embodiments, the Cu-MOR-IWI catalyst has a copper content of about 3.7 wt. % based on a total weight of the Cu-MOR-IWI catalyst as determined by EDX.

In some embodiments, the Cu-MOR-IWI catalyst has a methanol production yield of about 23.2 μmol of methanol per gram of the Cu-MOR-IWI catalyst in the first 10 minutes of contacting.

In some embodiments, the Cu-MOR-IWI catalyst comprises particles having a specific surface area in a range of 310 to 330 m²/g.

In some embodiments, the Cu-MOR-IWI catalyst comprises particles having a mesopore volume in a range of 0.1 to 0.15 cm³/g, and a micropore volume in a range of 0.16 to 0.18 cm³/g.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a flowchart depicting a method for direct methane (CH₄) oxidation (DMTM) to methanol (CH₃OH), according to certain embodiments;

FIG. 1B is a flowchart depicting a method for preparing the Cu-loaded mordenite zeolites by wetness impregnation, designated as Cu-MOR-WI, according to certain embodiments;

FIG. 2 shows a systematic route map for the synthesis of Cu-loaded mordenite zeolite (Cu-MOR) catalyst, according to certain embodiments;

FIG. 3 is a schematic illustration depicting setups for the direct methane oxidation to methanol, according to certain embodiments;

FIG. 4A shows a transmission electron microscope (TEM) image of copper-exchanged mordenite zeolite (MOR), according to certain embodiments;

FIG. 4B shows a TEM image of the Cu-MOR-WI catalyst, according to certain embodiments;

FIG. 4C shows a TEM image of the Cu-MOR catalyst prepared by loading Cu on Cu-MOR catalyst by incipient wet impregnation method, designated as Cu-MOR (IWI), according to certain embodiments;

FIG. 4D shows a TEM image of the Cu-MOR catalyst prepared by loading Cu on Cu-MOR by double solvent method, designated as Cu-MOR (DS), according to certain embodiments;

FIG. 4E shows a TEM image of the Cu-MOR catalyst prepared by loading Cu on Cu-MOR by physical mixing, designated as Cu-MOR (PM), according to certain embodiments;

FIG. 4F shows lattice fringes (d=0.24 nm) of the Cu-MOR (PM) obtained from a high-resolution transmission electron microscope (HRTEM), according to certain embodiments;

FIG. 5A shows a scanning electron microscope/energy-dispersive X-ray spectroscopy (SEM/EDS) mapping of Cu-MOR (WI), according to certain embodiments;

FIG. 5B shows an SEM/EDS mapping of Cu-MOR (DS), according to certain embodiments;

FIG. 5C shows an SEM/EDS mapping of Cu-MOR (IWI), according to certain embodiments;

FIG. 5D shows an SEM/EDS mapping of Cu-MOR (PM), according to certain embodiments;

FIG. 6A shows a N₂-adsorption-desorption profile of the Cu-MOR zeolites prepared by various methods, according to certain embodiments;

FIG. 6B shows a Barrett-Joyner-Halenda (BJH) pore size distribution graph of the Cu-MOR zeolites prepared by various methods, according to certain embodiments;

FIG. 7A shows an X-ray diffractogram (XRD) spectra of the Cu-MOR zeolites prepared by various methods, according to certain embodiments;

FIG. 7B shows a Fourier Transform infrared (FTIR) spectra of the Cu-MOR zeolites prepared by various methods, according to certain embodiments;

FIG. 8A shows a diffuse-reflectance ultraviolet-visible (DR UV-Vis) spectra of the Cu-MOR zeolites prepared by various methods, according to certain embodiments;

FIG. 8B shows a methane temperature programmed desorption (CH₄-TPD) profile for methane adsorption attraction of CH₄ with the Cu-MOR zeolites prepared by various methods, according to certain embodiments;

FIG. 9A illustrates a plot depicting a catalytic efficiency of the Cu-MOR zeolites prepared by various methods, according to certain embodiments;

FIG. 9B illustrates a plot depicting effect of the contact time of methane on the Cu-MOR (IWI) catalytic surface, according to certain embodiments;

FIG. 9C illustrates a plot depicting effect of activation temperature on methanol production, according to certain embodiments; and

FIG. 9D illustrates a plot depicting effect of extraction water volume (0.7 g catalyst per reaction batch) on methanol production, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

As used herein, the words “about,” “approximately,” or “substantially similar” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), or +/−20% of the stated value (or range of values). Within the description of this disclosure, where a numerical limit or range is stated, the endpoints are included unless stated otherwise. Also, all values and subranges within a numerical limit or range are specifically included as if explicitly written out.

Aspects of the present disclosure are directed to a highly active Cu-loaded mordenite zeolite (Cu-MOR) catalyst, also referred to as a catalyst, formed by hydrothermal and/or ion exchange processes. Different methods were employed for Cu incorporation in the catalyst via double solvent (DS), physical mixing (PM), wetness impregnation (WI), and incipient wetness impregnation (IWI). The synthesized catalysts were characterized by various analytical techniques and evaluated for effectiveness in direct conversion of methane to methanol (DMTM).

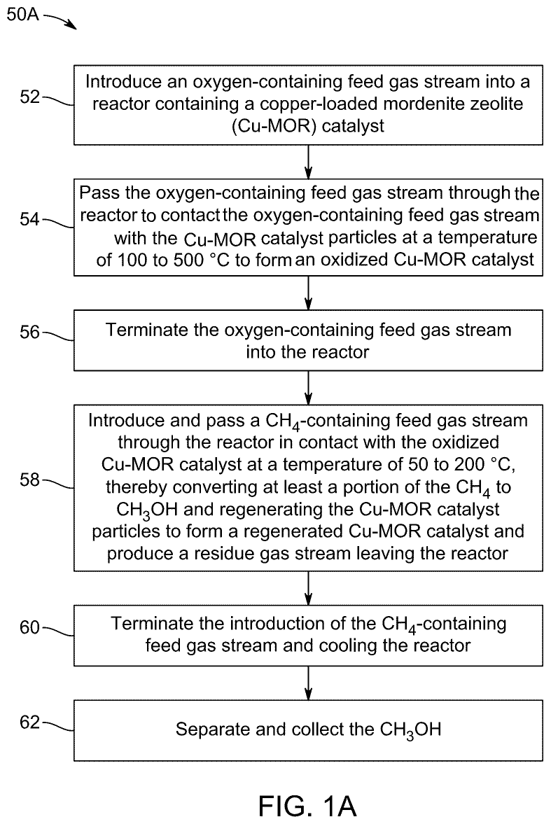

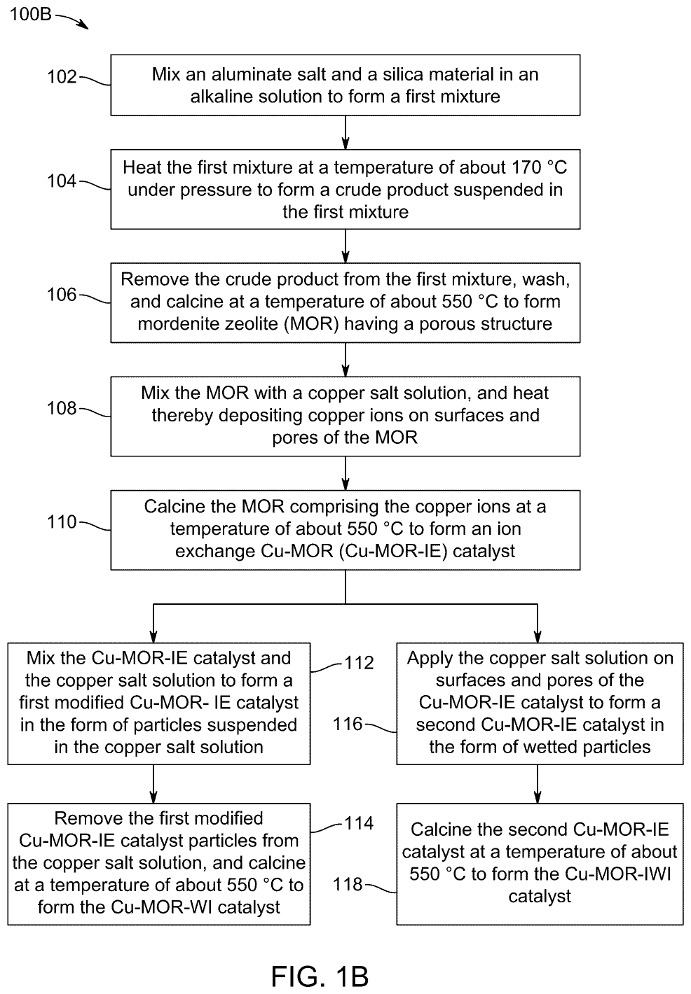

Referring to FIG. 1A, a method 50 for direct methane (CH₄) oxidation (DMTM) to methanol (CH₃OH) is described. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes introducing an oxygen-containing feed gas stream into a reactor containing a copper-loaded mordenite zeolite (Cu-MOR) catalyst. In this step, a feed gas stream rich in oxygen, particularly molecular oxygen, (e.g., including more than 95%, preferably 96%, preferably 97%, preferably 98%, preferably 99%, preferably 99.5%, preferably 99.9% of molecular oxygen) is fed into the reactor. In some embodiments, the oxygen-containing feed gas stream is introduced at a flow rate of 20-80 milliliters per minute (mL/min), preferably 30 mL/min, preferably 40 mL/min, preferably 50 mL/min, and was held constant for a period of 6-10 hours, preferably 7-9 hours, preferably 8 hours. Other ranges are also possible.

The Cu-MOR catalyst is pre-loaded into the reactor. The reactor may be a fixed-bed reactor, a trickle-bed reactor, a moving bed reactor, a rotating bed reactor, a fluidized bed reactor, or a slurry reactor. In a specific embodiment, the reactor is a fixed-bed reactor. In some embodiments, the fixed-bed reactor is in the form of a cylindrical reactor having a top portion, a cylindrical body portion, and a bottom portion. The fixed-bed reactor further includes a housing having an open top and open bottom supportably maintained with the cylindrical body portion. In some embodiments, the Cu-MOR catalyst is supportably retained within the housing, permitting fluid flow therethrough. In some embodiments, the reactor further includes at least one propeller agitator disposed in the bottom portion of the reactor. In some embodiments, the bottom portion is cone-shaped or pyramidal. In some embodiments, a plurality of recirculation tubes fluidly connects the bottom portion of the cylindrical reactor with the cylindrical body portion of the cylindrical reactor.

The catalytic activity of the Cu-MOR catalyst is significantly affected by the method adopted for the preparation of the Cu-MOR catalyst. The catalyst may be prepared by various methods, such as wetness impregnation (herein designated as Cu-MOR-WI catalyst) and/or by incipient wet impregnation (herein designated as Cu-MOR-IWI). In some embodiments, the Cu-MOR catalyst may be prepared by other methods known in the art—such as double solvent (herein designated as Cu-MOR-DS catalyst) or via physical mixing (herein designated as Cu-MOR-PM catalyst). The Cu-MOR catalyst includes particles having a porous structure; the size of the pores is dependent on the method of preparation of the Cu-MOR catalyst.

As used herein, the term “micropore” generally refers to small pores within a porous structure that have a diameter, e.g., preferably less than 10 nm, preferably less than 8 nm, preferably less than 6 nm, preferably less than 4 nm, or even more preferably less than 2 nm. Other ranges are also possible. In the present disclosure, the term “micropore volume” generally refers to a total volume of micropores.

As used herein, the term “mesopore” generally refers to pores within a porous structure that have an average diameter in a range of, e.g., preferably 2 to 100 nm, preferably 4 to 80 nm, preferably 6 to 60 nm, preferably 8 to 40 nm, preferably 10 to 20, or even more preferably about 15 nm. Other ranges are also possible. In the present disclosure, the term “mesopore volume” generally refers to a total volume of mesopores.

For example, a mesopore volume of the Cu-MOR-WI catalyst is in a range of 0.07-0.15 cm³/g, preferably 0.09 to 0.14 cm³/g, preferably 0.11 to 0.13 cm³/g, or even more preferably about 0.13 cm³/g. In some embodiments, a micropore volume of the Cu-MOR-WI catalyst is in a range of 0.10-0.18 cm³/g, preferably 0.12 to 0.17 cm³/g, preferably 0.14 to 0.16 cm³/g, or even more preferably about 0.16 cm³/g. In some embodiments, the mesopore volume of the Cu-MOR-IWI catalyst is in a range of 0.07-0.15 cm³/g, preferably 0.09 to 0.14 cm³/g, preferably 0.11 to 0.13 cm³/g, or even more preferably about 0.13 cm³/g. In some embodiments, the micropore volume of the Cu-MOR-IWI catalyst is in a range of 0.10-0.20 cm³/g, preferably 0.12 to 0.19 cm³/g, preferably 0.14 to 0.18 cm³/g, or even more preferably about 0.17 cm³/g. In some embodiments, the mesopore volume of the Cu-MOR-PM catalyst is in a range of 0.1-0.20 cm³/g, preferably 0.12 to 0.19 cm³/g, preferably 0.14 to 0.18 cm³/g, or even more preferably about 0.17 cm³/g. In some embodiments, the micropore volume of the Cu-MOR-PM catalyst is in a range of 0.1-0.20 cm³/g, preferably 0.12 to 0.19 cm³/g, preferably 0.14 to 0.18 cm³/g, or even more preferably about 0.17 cm³/g. In some embodiments, the mesopore volume of the Cu-MOR-DS catalyst is in a range of 0.05-0.16 cm³/g, preferably 0.07 to 0.14 cm³/g, preferably 0.09 to 0.12 cm³/g, or even more preferably about 0.11 cm³/g. In some embodiments, the micropore volume of the Cu-MOR-DS catalyst is in a range of 0.1-0.18 cm³/g, preferably 0.12 to 0.17 cm³/g, preferably 0.14 to 0.16 cm³/g, or even more preferably about 0.15 cm³/g. The particles have an average particle size of 100 to 800 micrometers (μm), preferably 150 to 600 μm, preferably 200 to 400 μm, or even more preferably 250 to 300 μm. Other ranges are also possible.

At step 54, the method 50 includes passing the oxygen-containing feed gas stream through the reactor to contact the oxygen-containing feed gas stream with the Cu-MOR catalyst particles at a temperature of 50 to 800° C., preferably 100 to 600° C., preferably 150 to 400° C., or even more preferably about 300° C. to form an oxidized Cu-MOR catalyst. The Cu-MOR catalyst particles are activated upon contacting the oxygen-containing feed gas stream that is rich in molecular oxygen, to form the oxidized Cu-MOR catalyst. The activation of the Cu-MOR catalyst particles may be carried out in a furnace, such as an electric furnace or any other form of such containment. In some embodiments, the furnace is preferably equipped with a temperature control system, which may provide a heating rate of up to 50° C./min, or preferably up to 40° C./min, or preferably up to 30° C./min, preferably up to 20° C./min, preferably up to 10° C./min, preferably up to 5° C./min, preferably up to 3° C./min, preferably up to 2° C./min, till the temperature in the furnace reaches about 100-500° C. Other ranges are also possible.

At step 56, the method 50 includes terminating the introducing the oxygen-containing feed gas stream. Once the catalyst is activated, or in other words, once the oxidized Cu-MOR catalyst is formed, the supply of the oxygen-containing feed gas stream into the reactor is terminated. Generally, the supply of the oxygen-containing feed gas stream into the reactor is terminated after about 6-10 hours, preferably 7-9 hours, and yet more preferably after 8 hours of passing the oxygen-containing feed gas stream into the reactor. Other ranges are also possible. Once the supply of oxygen is terminated, the oxygen in the reactor is displaced by purging nitrogen into the reactor. During this process, nitrogen is purged into the reactor for 1-10 minutes, preferably 2-9 minutes, preferably 3-8 minutes, preferably 4-7 minutes, preferably 5-6 minutes, preferably 5 minutes to completely displace oxygen and other gases from the reactor without reacting chemically with the Cu-MOR catalyst. Other ranges are also possible. In some embodiments, an inert gas, such as helium or argon, may be purged as well, alone or in combination with nitrogen, into the reactor to displace the oxygen in the reactor.

At step 58, the method 50 includes introducing and passing a CH₄-containing feed gas stream through the reactor to contact the CH₄-containing feed gas stream with the oxidized Cu-MOR catalyst at a temperature of 30 to 300° C., preferably 50 to 250° C., preferably 100 to 200° C., or even more preferably about 150° C. thereby converting at least a portion of the CH₄ to CH₃OH and regenerating the Cu-MOR catalyst particles to form a regenerated Cu-MOR catalyst and producing a residue gas stream leaving the reactor. In some embodiments, the CH₄-containing feed gas stream is introduced and passed through the reactor at a flow rate of 10 to 100 mL/min, preferably 20 to 80 mL/min, preferably 30 to 60 mL/min, or even more preferably 35 to 40 mL/min. Other ranges are also possible. In some embodiments, the CH₄-containing feed gas stream predominantly includes methane (greater than 50 vol. %, preferably 60 vol. %, preferably 70 vol. %, preferably 80 vol. %, preferably 90 vol. %, preferably 95 vol. %, each vol. % based on a total volume of the CH₄-containing feed gas stream). Other ranges are also possible. In some embodiments, the CH₄-containing feed gas stream may also include inert gases, such as nitrogen, argon, and helium. In a specific embodiment, the CH₄-containing feed gas stream includes methane and nitrogen. In some preferred embodiments, a volume ratio of the methane to nitrogen present in the CH₄-containing feed gas stream is in a range of 20:1 to 1:1, preferably 15:1 to 2:1, preferably 10:1 to 3:1, or even more preferably about 6:1. Other ranges are also possible.

The concentration of the inert gas in the CH₄-containing feed gas stream may be in the range of 1-50 vol. %, preferably 2-45 vol. %, preferably 3-40 vol. %, preferably 4-35 vol. %, preferably 5-30 vol. %, and yet more preferably 6-25 vol. %, each vol. % based on a total volume of the CH₄-containing feed gas stream. In an embodiment, the concentration of CH₄ in the CH₄-containing feed gas stream may be in the range of 5 to 50 vol. % based on the total volume of the CH₄-containing feed gas stream. The volume ratio of the CH₄ to the inert gas present in the CH₄-containing feed gas stream is in a range of 1:1 to 1:20, preferably 1:2 to 1:18, preferably 1:3 to 1:15, preferably 1:4 to 1:10, preferably 1:5 to 1:8, preferably 1:6. Other ranges are also possible.

Once the CH₄-containing feed gas stream is introduced into the reactor, the methane reacts with the oxidized Cu-MOR catalyst, in an oxygen-free atmosphere, where at least a portion of methane is converted to methanol or methanol precursors. In a preferred embodiment, the reaction is carried out at 200° C. The methanol formed during the reaction is adsorbed on the surfaces and pores of the regenerated Cu-MOR catalyst. A number of gases, such as methane, nitrogen, formaldehyde, carbon monoxide, carbon dioxide, and nitrogen oxides (NOₓ), may be formed as by-products during this chemical reaction. In some embodiments, the NOₓ includes at least one of nitric oxide (NO), nitrogen dioxide (NO₂), nitrous oxide (N₂O), dinitrogen tetroxide (N₂O₄), and dinitrogen pentoxide (N₂O₅). These gases, which form the residue gas stream, leave the reactor.

At step 60, the method 50 includes terminating the introduction the CH₄-containing feed gas stream and cooling the reactor. The supply of the CH₄-containing feed gas stream is terminated about 60 minutes after the introduction of the CH₄-containing feed gas stream into the reactor. Other ranges are also possible. The reactor is further cooled to room temperature for methanol recovery.

At step 62, the method 50 includes separating and collecting the CH₃OH. The methanol can be extracted or collected from the pores of the regenerated Cu-MOR catalyst by any of the conventional methods known in the art. In a specific embodiment, the methanol is extracted offline with liquid water. More specifically, the regenerated Cu-MOR catalyst adsorbed with methanol is dispersed into an aqueous solution (water) and stirred for 1-3 hours, preferably 2 hours. Other ranges are also possible. The stirring aids in the desorption of methanol into water. The catalyst is further dried and re-generated for re-use. The method results in having a methanol production yield of about 5 to 50 micro-mole (μmol) of methanol per gram of the Cu-MOR catalyst, preferably 10 to 30, or even more preferably about 20 μmol of methanol per gram of the Cu-MOR catalyst in the first 5 to 20 minutes, or even more preferably in the first 10 minutes of contacting.

Referring to FIG. 1B, a method 100 for preparing Cu-MOR-WI catalyst, is described. The order in which the method 100 is described is not intended to be construed as a limitation, and any number of the described method steps can be combined in any order to implement the method 100. Additionally, individual steps may be removed or skipped from the method 100 without departing from the spirit and scope of the present disclosure.

At step 102, the method 100 includes mixing an aluminate salt and a silica material in an alkaline solution to form a first mixture. In some embodiments, the aluminate salt is at least one of sodium aluminate and/or potassium aluminate. In a preferred embodiment, the aluminate salt is sodium aluminate. The silica may be used in the form of sodium silicate or silica gel. In some embodiments, a weight ratio of the aluminate salt to the silica material is in a range of 1:1 to 1:10, preferably 1:2 to 1:9, preferably 1:3 to 1:8, preferably 1:4 to 1:7, preferably 1:5 to 1:7, preferably 1:6 to 1:7. Other ranges are also possible. However, this weight ratio can be adjusted based on the desired SiO₂/Al₂O₃ ratio. The aluminate salt and a silica material are mixed in the alkaline solution to form the first mixture. In some embodiments, the first mixture is formed by dissolving a base such as sodium hydroxide, potassium hydroxide, calcium hydroxide, or the like in water. In a preferred embodiment, the base is sodium hydroxide.

At step 104, the method 100 includes heating the first mixture at a temperature of about 210° C., preferably about 190° C., or even more preferably about 170° C. under pressure to form a crude product suspended in the first mixture. The first mixture is kept in the autoclave for a hydrothermal reaction. The reaction is carried out for 20-30 hours, preferably 22-26 hours, preferably 24 hours, to form the crude product.

At step 106, the method 100 includes removing the crude product from the first mixture, washing, and calcining at a temperature of about 550° C. to form mordenite zeolite (MOR) having a porous structure. The crude product is filtered, and washed to remove any unreacted salts or impurities, and further calcined for 5-7 hours, preferably 6 h at 550° C. Other ranges are also possible. The calcination is carried out in a furnace, such as an electric furnace or any other form of such containment. The furnace is preferably equipped with a temperature control system, which may provide a heating rate of up to 50° C./min, or preferably up to 40° C./min, or preferably up to 30° C./min, preferably up to 20° C./min, preferably up to 10° C./min, preferably up to 5° C./min, preferably up to 3° C./min, till the temperature in the furnace reaches about 550° C. Other ranges are also possible. The calcination results in the formation of a porous MOR.

At step 108, the method 100 includes mixing the MOR with a copper salt solution, and heating thereby depositing copper ions on surfaces and pores of the MOR. In an embodiment, the copper salt solution includes a copper salt selected from copper sulfate, copper nitrate, copper chloride, copper acetate, copper carbonate, copper phosphate, and/or a hydrate thereof. In a preferred embodiment, the copper salt is copper nitrate. In some embodiments, the concentration of the copper salt in the copper salt solution is in a range of 0.01 to 1 M, preferably 0.02 M, preferably 0.03 M, preferably 0.04 M, and yet more preferably 0.05 M. Other ranges are also possible. Preferably, a stirrer may be used for mixing the MOR with the copper salt solution to ensure complete mixing and deposition of the copper ions on the surface and the pores of the MOR. The mixing is carried out for a period of 4-24 hours, preferably 6 to 18 hours, or even more preferably 8-12 hours. Other ranges are also possible.

At step 110, the method 100 includes calcining the MOR comprising the copper ions at a temperature of about 400 to 800° C., preferably 500 to 700° C., or even more preferably about 550° C. to form an ion exchange Cu-MOR (Cu-MOR-IE) catalyst. The product obtained in step 108 is calcined at 550° C. for 2-6 hours, preferably 3-5 hours, preferably 4 hours, to form the ion exchange Cu-MOR (Cu-MOR-IE) catalyst. The Cu-MOR-IE catalyst has a copper content of about 1 to 5 wt. %, preferably 1.5 to 4.5 wt. %, preferably 2 to 4 wt. %, or even more preferably about 2.5 wt. % based on the total weight of the Cu-MOR-IE catalyst as determined by energy-dispersive X-ray spectroscopy (EDX). When used as a catalyst for methanol production, the Cu-MOR-IE catalyst has a methanol production yield of about 12.4 μmol of methanol per gram of the Cu-MOR-IE catalyst in the first 5 to 20 minutes, or even more preferably in the first 10 minutes of contacting.

At step 112, the method 100 includes mixing the Cu-MOR-IE catalyst and the copper salt solution to form a first modified Cu-MOR-IE catalyst in the form of particles suspended in the copper salt solution. In an embodiment, the copper salt solution includes a copper salt selected from copper sulfate, copper nitrate, copper chloride, copper acetate, copper carbonate, copper phosphate, and/or a hydrate thereof. In a preferred embodiment, the copper salt is copper nitrate. In some embodiments, the concentration of the copper salt in the copper salt solution is in a range of 0.01 to 1 M, preferably 0.02 M, preferably 0.03 M, preferably 0.04 M, and yet more preferably 0.05 M. Other ranges are also possible.

At step 114, the method 100 includes removing the first modified Cu-MOR-IE particles from the copper salt solution, and calcining at a temperature of about 550° C. to form the Cu-MOR-WI catalyst. In some embodiments, the Cu-MOR-WI catalyst particles are removed from the salt solution and dried for 2-4 hours, preferably 3 hours, and dried. In some embodiments, the calcining may be performed at a temperature of about 400 to 800° C., preferably 500 to 700° C., or even more preferably about 550° C. to form the Cu-MOR-WI catalyst. The Cu-MOR-WI catalyst includes particles having a specific surface area in a range of 290 to 300 square meters per gram (m²/g), preferably about 294-298 m²/g, and yet more preferably about 296 m²/g. Other ranges are also possible. In some embodiments, the Cu-MOR-WI catalyst includes a mesopore volume in a range of 0.1 to 0.15, preferably 0.12 to 0.14, preferably 0.13 cm³/g. In some embodiments, the Cu-MOR-WI catalyst includes a micropore volume in a range of 0.15 to 0.17, preferably 0.16 cm³/g. In some embodiments, the Cu-MOR-WI catalyst includes CuO nanoparticles having an average particle size of 3 to 10 nm, preferably 5 to 9 nm, or even more preferably about 7 nm. In some embodiments, the Cu-MOR-WI catalyst has a copper content of about 2 to 5 wt. %, or preferably about 3.7 wt. % based on the total weight of the Cu-MOR-WI catalyst as determined by EDX, and a methanol production yield of about 26.5 μmol of methanol per gram of the Cu-MOR-WI catalyst in the first 5 to 20 minutes, or even more preferably in the first 10 minutes of contacting. Other ranges are also possible.

Also referring to FIG. 1B, the method 100 further comprises preparing the Cu-MOR-IWI catalyst. At step 116, the method 100 includes applying the copper salt solution on surfaces and pores of the Cu-MOR-IE catalyst to form a second Cu-MOR-IE catalyst in the form of wetted particles. In some embodiments, the applying is performed by dropwise adding the copper salt solution on particles of the Cu-MOR-IE catalyst. The copper salt solution applied may be absorbed via capillary action into the surfaces and pores of the Cu-MOR-IE catalyst without assistance of external forces, such as gravity. The capillary action may be achieved due to the effects of adhesive and cohesive forces between interface of the copper salt solution and the the Cu-MOR-IE catalyst. Initially the copper salt solution adheres to the surface of the Cu-MOR-IE catalyst due to adhesive forces. The copper ions in the solution are attracted to the surface of the Cu-MOR-IE catalyst, forming a thin layer of liquid. In some further embodiments, the copper salt solution applied may be diffused into the voids of the Cu-MOR-IE catalyst through the interconnected network of voids within the Cu-MOR-IE catalyst. This diffusion is a result of the random motion of the copper ions and solvent molecules. They move from areas of higher concentration to areas of lower concentration, therefore achieving equilibrium throughout the structure of the Cu-MOR-IE catalyst. In some embodiments, without separating, the second Cu-MOR-IE catalyst may be dried for 2-4 hours, preferably 3 hours, at room temperature.

At step 116, the method 100 includes calcining the second Cu-MOR-IE at a temperature of about 400 to 800° C., preferably 500 to 700° C., or even more preferably about 550° C. to form the Cu-MOR-IWI catalyst comprising CuO nanoparticles having an average particle size of 7 to 15 nm, preferably 8 to 13 nm, or even more preferably about 9 nm to prepare the Cu-MOR-IWI catalyst. In some embodiments, the Cu-MOR-IWI catalyst includes particles having a specific surface area in a range of 310 to 330, preferably 315 to 325, preferably 320 m²/g. In some embodiments, the Cu-MOR-IWI catalyst has a mesopore volume in a range of 0.1 to 0.15, preferably 0.12 to 0.14, preferably 0.13 cm³/g. In some embodiments, the Cu-MOR-IWI catalyst includes a micropore volume in a range of 0.16 to 0.18, preferably 0.17 cm³/g. In some embodiments, the Cu-MOR-IWI catalyst has a copper content of about 2 to 5 wt. %, or preferably about 3.7 wt. % based on the total weight of the Cu-MOR-IWI catalyst as determined by EDX. In some embodiments, the Cu-MOR-IWI catalyst has a methanol production yield of about 23.2 μmol of methanol per gram of the Cu-MOR-IWI catalyst in the first 5 to 20 minutes, or even more preferably in the first 10 minutes of contacting.

As used herein, the term “N₂ adsorption/desorption method” generally refers to a technique used to measure the specific surface area of a solid material, such as an adsorbent material or a porous material. In some embodiments, the Cu-MOR catalysts, including Cu-MOR-DS, Cu-MOR-WI, Cu-MOR-IWI, and Cu-MOR-PM catalyst, are exposed to a stream of nitrogen gas at low temperature and pressure, respectively. The nitrogen gas is adsorbed onto the surface of the Cu-MOR catalyst, filling the pores and creating a monolayer of adsorbed nitrogen. In some further embodiments, the amount of nitrogen adsorbed at a given pressure is measured using a gas adsorption instrument, such as a BELCAT II analyzer. In some preferred embodiments, the BET analysis is performed on the BELCAT II analyzer according to the software manual. In some more preferred embodiments, the nitrogen gas is gradually removed from the Cu-MOR catalyst, causing the desorption of the adsorbed nitrogen. The amount of nitrogen desorbed at a given pressure is also measured using the gas adsorption instrument. By analyzing the amount of nitrogen adsorbed and desorbed, the specific surface area of the Cu-MOR catalyst can be calculated using the BJH (Brunauer-Emmett-Teller) and Barrett, Joyner and Halenda (BJH) equation.

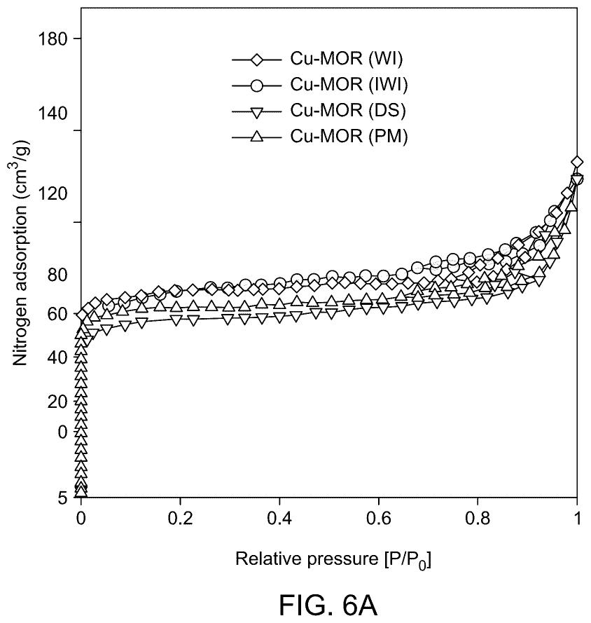

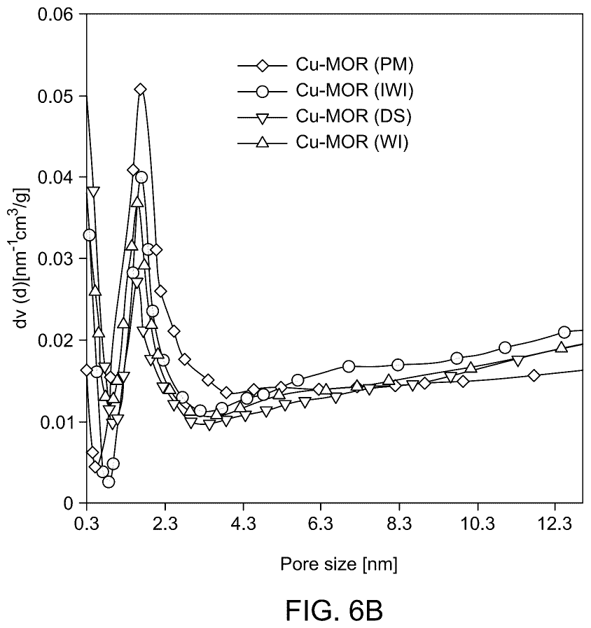

Referring to FIG. 6A, the Cu-MOR-PM has a specific surface area in a range of 320 to 350, preferably 325 to 345, preferably 335 m²/g. In some embodiments, the Cu-MOR-DS has a specific surface area in a range of 295 to 325, preferably 305 to 315, preferably 310 m²/g. In some embodiments, the Cu-MOR-WI has a specific surface area in a range of 280 to 310, preferably 290 to 300, preferably 296 m²/g. In some embodiments, the Cu-MOR-IWI has a specific surface area in a range of 305 to 335, preferably 315 to 325, preferably 320 m²/g. Other ranges are also possible.

Referring to FIG. 6B, the Cu-MOR-PM has a micropore volume in a range of 0.13 to 0.21 square centimeters per gram (cm²/g), preferably 0.15 to 0.19 cm²/g, or even more preferably about 0.17 cm²/g. In some embodiments, the Cu-MOR-DS a micropore volume in a range of 0.11 to 0.19 cm²/g, preferably 0.13 to 0.17 cm²/g, or even more preferably about 0.15 cm²/g. In some embodiments, the Cu-MOR-WI has a micropore volume in a range of 0.12 to 0.20 cm²/g, preferably 0.14 to 0.18 cm²/g, or even more preferably about 0.16 cm²/g. In some embodiments, the Cu-MOR-IWI has a micropore volume in a range of 0.13 to 0.21 cm²/g, preferably 0.15 to 0.19 cm²/g, or even more preferably about 0.17 cm²/g. Other ranges are also possible.

The crystalline structures of the Cu-MOR catalysts, including Cu-MOR-DS, Cu-MOR-WI, Cu-MOR-IWI, and Cu-MOR-PM catalyst, may be characterized by X-ray diffraction (XRD). The XRD patterns are collected in a Mini-Flex II bench-top diffractometer equipped with a Cu—Kα radiation source (λ=0.15406 nm) for a 20 range extending between 5 and 100°, preferably 15 and 80°, further preferably 30 and 60° at an angular rate of 0.005 to 0.04° s⁻¹, preferably 0.01 to 0.03° s⁻¹ or even preferably 0.02° s⁻¹.

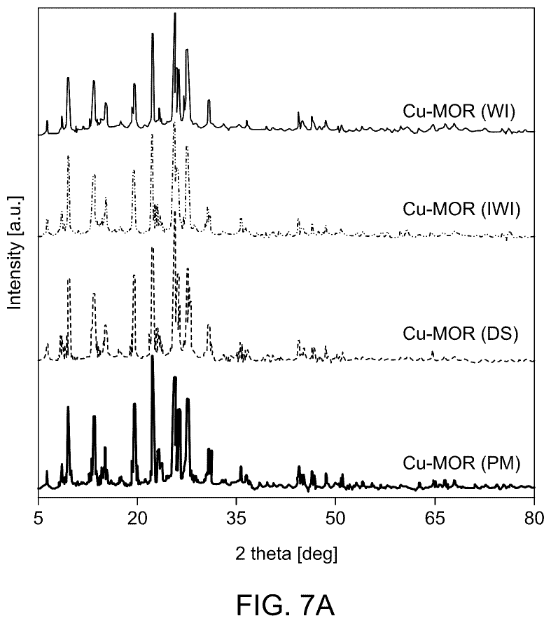

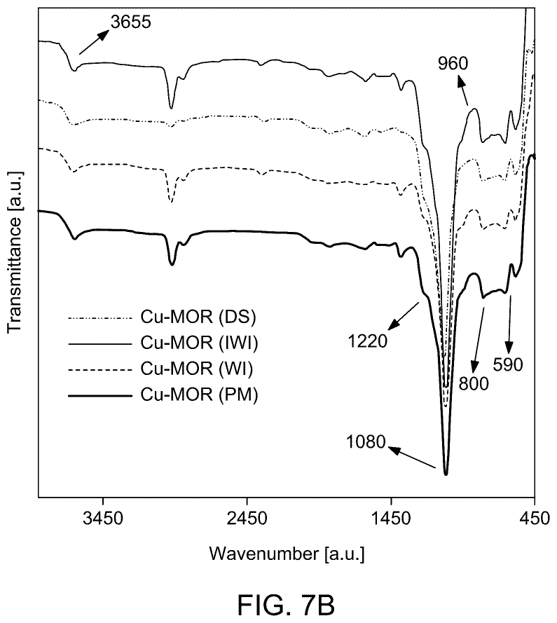

Referring to FIG. 7A, XRD profiles for Cu-MOR catalysts prepared with different methods. In some embodiments, the Cu-MOR-PM catalyst has at least a first intense peak with a 2 theta (θ) value in a range of 5 to 20°, preferably about 10 to 15°; at least a second intense peak with a 2θ value in a range of 20 to 25°, preferably about 22°; at least a third intense peak with a 2θ value in a range of 25 to 40°, preferably about 27 to 32°, as depicted in FIG. 7A. In some embodiments, the Cu-MOR-DS catalyst has at least a first intense peak with a 2 theta (θ) value in a range of 5 to 19°, preferably about 10 to 15°; at least a second intense peak with a 2θ value in a range of 19 to 25°, preferably about 22°; at least a third intense peak with a 2θ value in a range of 25 to 40°, preferably about 27 to 32°, as depicted in FIG. 7A. In some embodiments, the Cu-MOR-WI catalyst has at least a first intense peak with a 2 theta (θ) value in a range of 5 to 15°, preferably about 10 to 15°; at least a second intense peak with a 2θ value in a range of 15 to 30°, preferably about 18 to 25°; at least a third intense peak with a 2θ value in a range of 30 to 40°, preferably about 35°, as depicted in FIG. 7A. In some embodiments, the Cu-MOR-IWI catalyst has at least a first intense peak with a 2 theta (θ) value in a range of 5 to 15°, preferably about 10 to 15°; at least a second intense peak with a 2θ value in a range of 15 to 30°, preferably about 18 to 25°; at least a third intense peak with a 2θ value in a range of 30 to 40°, preferably about 35° as depicted in FIG. 7A.

As used herein, the term “temperature program desorption using methane,” or “CH₄-TPD” generally refers to a technique used to study the surface acidity of a solid material, such as the Cu-MOR catalyst. In some embodiments, the Cu-MOR catalyst is first heated in an inert gas, such as nitrogen, to remove any adsorbed species and to stabilize the surface. In some embodiments, the Cu-MOR catalyst is then cooled down and exposed to a stream of methane gas, which is adsorbed onto the surface of the adsorbent material. The amount of methane adsorbed is proportional to the surface basicity of the Cu-MOR catalyst. In some embodiments, the Cu-MOR catalyst is then heated at a constant rate while the amount of methane desorbed is monitored as a function of temperature. In some further embodiments, as the temperature increases, the adsorbed methane begins to desorb from the surface of the Cu-MOR catalyst. In some preferred embodiments, the desorption of methane may be exothermic, and the heat generated by the desorption process is monitored using a thermal conductivity detector.

The Cu-MOR catalysts, including Cu-MOR-DS, Cu-MOR-WI, Cu-MOR-IWI, and Cu-MOR-PM catalyst, may be characterized mainly using CH₄-TPD. Temperature programmed desorption (TPD) is a technique used to monitor surface interactions between molecular species on a surface when the surface temperature has changed in a controlled setting. This is done by placing the Cu-MOR catalyst inside a reactor and pushing an inert gas into the chamber. Alternatively, the sample can be located in an ultra-high vacuum (UHV) chamber with no carrier gas. The sample is dosed with a probe gas such as CO, CH₄, H₂, etc. The sample is then increased in temperature at a linear ramp rate, and the desorption products are analyzed by a mass spectrometer.

The CH₄-TPD may be conducted on an Autochem II 2920 chemisorption analyzer. The Cu-MOR catalyst was heated at a temperature of 100 to 800° C., preferably about 100 to 750° C. under a gas flow of helium and hydrogen for 30 to 180 min, preferably about 120 min at a flow rate of 30 to 70 milliliters per minute (mL/min), preferably 50 mL/min. Other ranges are also possible. In some further embodiments, the Cu-MOR catalyst was then cooled to less than 300° C., preferably less than 250° C. before contacting with a NH₃-containing gas mixture. In some preferred embodiments, CH₄ is present in the gas mixture at a concentration of 1 to 20%, preferably about 5 to 15%, or even more preferably about 10% by volume. In some more preferred embodiments, the Autochem II 2920 chemisorption analyzer containing the Cu-MOR catalyst is heated to a temperature of 600 to 900° C., preferably about 750° C. at a heating rate of 5 to 30° C./min, preferably 5 to 20° C./min, or even more preferably about 10° C./min. Other ranges are also possible.

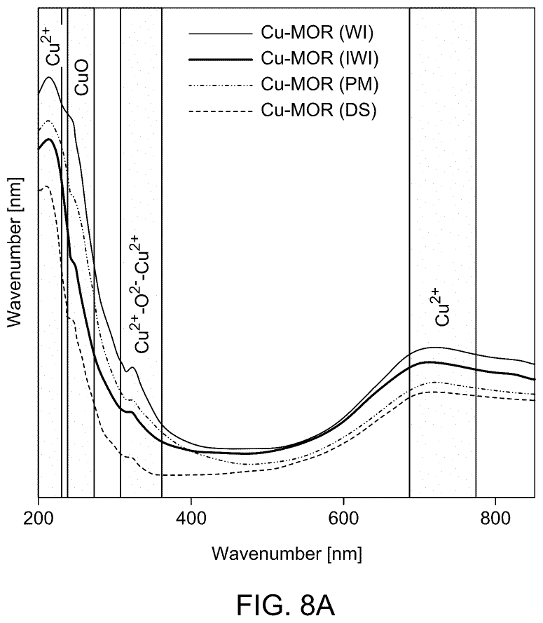

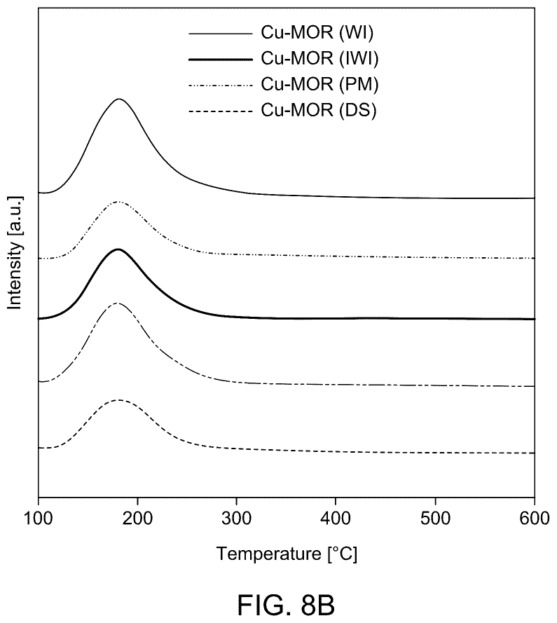

In some embodiments, the Cu-MOR-PM catalyst has a methane temperature-programmed desorption (CH₄-TPD) at a temperature in a range of 120 to 250° C., preferably 160 to 220° C., as depicted in FIG. 8B. In some embodiments, the Cu-MOR-DS catalyst has a CH₄-TPD at a temperature in a range of 120 to 250° C., preferably 140 to 200° C., as depicted in FIG. 8B. In some embodiments, the Cu-MOR-WI catalyst has a CH₄-TPD at a temperature in a range of 120 to 250° C., preferably 180 to 200° C., as depicted in FIG. 8B. In some embodiments, the Cu-MOR-IWI catalyst has a CH₄-TPD at a temperature in a range of 120 to 250° C., preferably 160 to 200° C., as depicted in FIG. 8B. Other ranges are also possible.

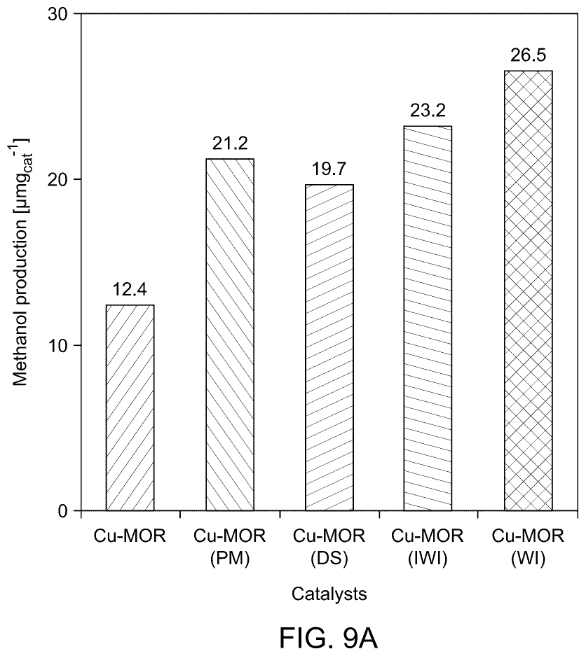

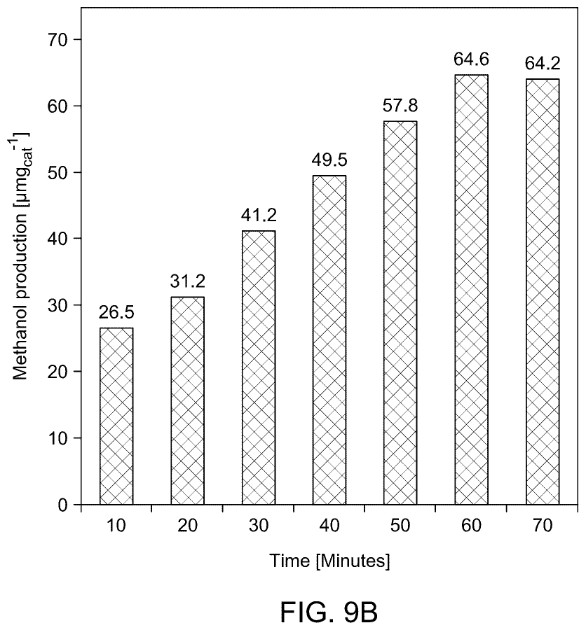

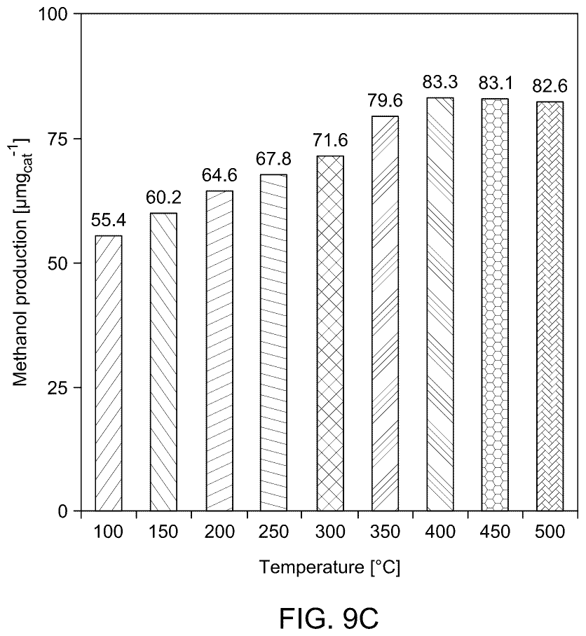

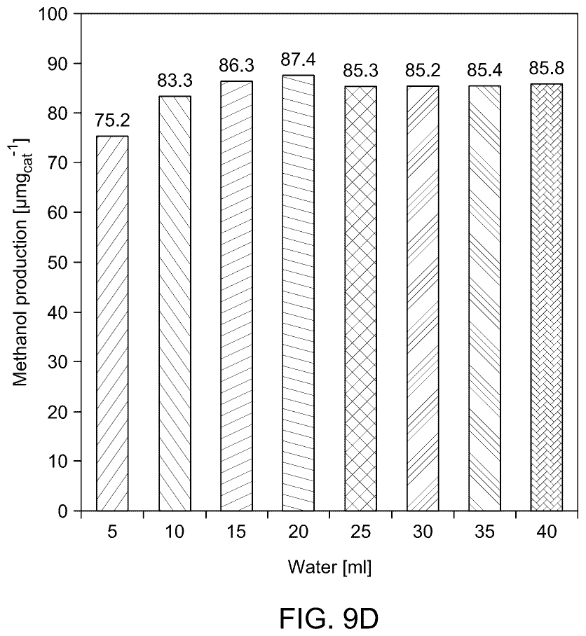

Referring to FIG. 9B, when the Cu-MOR catalyst is Cu-MOR-WI catalyst, the method has a methanol production yield of about 25 to 80 μmol per gram of the Cu-MOR-WI catalyst, preferably 30 to 70, or even more preferably 40 to 60 μmol per gram of the Cu-MOR-WI catalyst in a time period of 10 to 120 minutes, preferably 30 to 90 minutes, or even more preferably about 60 minutes. Other ranges are also possible.

Referring to FIG. 9C, when the Cu-MOR catalyst is Cu-MOR-WI catalyst, the method has a methanol production yield of about 40 to 100 μmol per gram of the Cu-MOR-WI catalyst, preferably 50 to 90, or even more preferably 60 to 80 μmol per gram of the Cu-MOR-WI catalyst at a temperature of 50 to 800° C., preferably 100 to 500° C., or even more preferably 200 to 400° C. Other ranges are also possible.

In some embodiments, the Cu-MOR catalyst prepared by wetness impregnation (Cu-MOR-WI) exhibited an increased methanol yield (e.g., about 26.5 μmol gcat⁻¹) compared to the catalysts prepared by other methods as a result of the high metal dispersion and high methane adsorption. This performance was related to the particle sizes of copper and the method of preparation. Additionally, it was also disclosed that the longer the methane was in contact with the Cu-MOR-WI catalyst, the greater the amount of methanol that was produced (e.g., about 83.3 μmol gcat⁻¹).

### EXAMPLES

The following examples describe and demonstrate exemplary embodiments as described herein. The examples are provided solely for the purpose of illustration and are not to be construed as limitations of the present disclosure, as many variations thereof are possible without departing from the spirit and scope of the present disclosure.

### Example 1: Catalyst Preparation

In a typical procedure, about 19 g sodium hydroxide was added to distilled water stir until dissolved, followed by the addition of about 14.31 g sodium aluminate. The solution was stirred for 30 min with 645 g purified water and 98.2 g silica. The required mixture was transferred into a Teflon-lined stainless-steel autoclave and kept at 170° C. for 24 h. The required product was recovered by filtering and washing to pH<10. The solid sample was dried at 100° C. The solid product was then ground into a fine powder and calcined for 6 h at 550° C. with a ramping rate of 3° C./min in an electric furnace. The catalyst that was synthesized was designated as “MOR” (G. J. Kim, W. S. Ahn, Direct synthesis and characterization of high-SiO₂-content mordenites, Zeolites 11 (7) (1991) 745-750, which is incorporated herein by reference in its entirety).

### Example 2: Synthesis of Cu-Exchanged and Cu-Loaded MOR

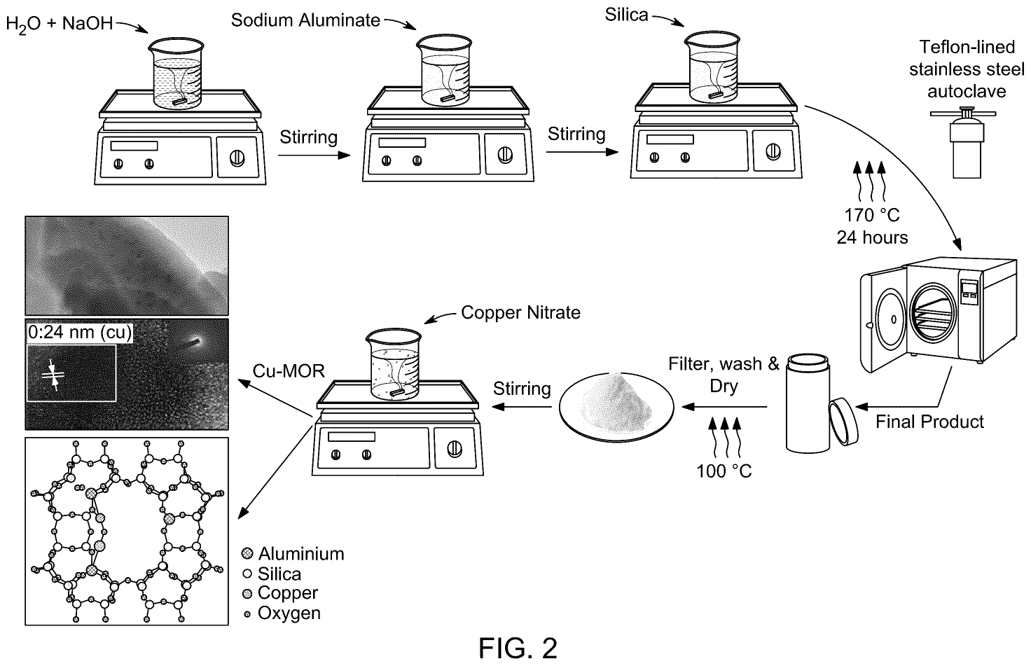

Typically, about 5 g of MOR was added to 0.5 L of a 0.05 M aqueous solution of copper nitrate (99%, Sigma-Aldrich) at 50° C. and mixed overnight (A. M. Rabie, M. A. Betiha, S. E. Park, Direct synthesis of acetic acid by simultaneous co-activation of methane and CO2 over Cu-exchanged ZSM-5 catalysts, Appl. Catal. B Environ. 215 (2017) 50-59, which is incorporated herein by reference in its entirety). The Cu ion exchange procedure was repeated thrice using the same method. After filtration, it was washed and dried at 100° C. for an hour; the as-synthesized materials were calcined at 550° C. for 4 h in dry air. The samples were designated as Cu-MOR and energy-dispersive X-ray spectroscopy (EDX) was used to measure the Cu content of the samples. Additionally, Cu was loaded on Cu-MOR using different methods, for example, double-solvent (DS), physical mixing (PM), incipient wet impregnation (IWI), and wetness impregnation (WI) methods. The Cu-MOR (PM) was prepared by physically grinding the Cu precursor, and Cu exchanged MOR support in a pestle for 20 min; the resultant powder was heated (at 550° C. for 3 h) (P. J. Miedziak, S. A. Kondrat, N. Sajjad, G. M. King, M. Douthwaite, G. Shaw, G. L. Brett, J. K. Edwards, D. J. Morgan, G. Hussain, G. J. Hutchings, Physical mixing of metal acetates: optimization of catalyst parameters to produce highly active bimetallic catalysts, Catal. Sci. Technol. 3 (11) (2013) 2910-2917, which is incorporated herein by reference in its entirety). The Cu-MOR support was suspended in cyclohexane liquid to create the Cu-MOR (DS) catalyst. After 2 h of rapid stirring at 25° C., the aqueous Cu precursor solution was dropwise added to the suspension. The mixture was constantly agitated (3 h), dried (12 h), and autoclaved (3 h at 550° C.) (M. Tao, Z. Xin, X. Meng, Y. Lv, Z. Bian, Impact of double-solvent impregnation on the Ni dispersion of Ni/SBA-15 catalysts and catalytic performance for the syngas methanation reaction, RSC Adv. 6 (42) (2016) 35875-35883, which is incorporated herein by reference in its entirety]. The Cu-MOR (WI) catalyst was synthesized in the absence of cyclohexane. In porous Cu-MOR, an adequate Cu-containing aqueous solution of Cu (NO₃)₂·3H₂O was prepared with the addition of Cu-MOR as a support solution. The mixture was dried and preserved in its natural environment. The catalyst was then calcined in a static environment for 3 h at 550° C. The produced catalyst was designated as Cu-MOR (IWI); a typical procedure is shown in FIG. 2.

### Example 3: Characterization of the Materials

An EDAX Energy-dispersive X-ray spectroscopy (EDX) system (Si (Li)) SUTW detector, with an energy resolution of 136 eV (for Mn K radiation), was coupled to a TECNAI Model G2 20 STWIN electron microscope operating at 200 kV to obtain transmission electron microscope (TEM) and EDX measurements. A sample was prepared by evaporating one drop of the substance suspended in ethanol onto a carbon-coated nickel grid. A QUADRASORB SI system with an automated surface area analyzer analyzed N₂ sorption at 77 K. Samples were evaporated to dryness at 300° C. for 3 h before evaluation. The particular surface areas (BET) were estimated over the P/P₀=0-1. The Barrett-Joyner-Halenda (BJH) study determined pore size distributions and the t-plot method estimated microporous volume. Powder X-ray Diffraction (XRD) using Cu Kα radiation (1.5418 Å), a Bruker-AXS Model D8 Advanced diffractometer with DAVINCI design, and a Lynx Eye detector conducted wide-angle powder XRD. A Cary Model 5000 spectrometer (Agilent, U.S.A) with a Harrick Praying Mantis diffuse reflectance attachment (Model DRPP72) and a reaction chamber recorded UV-visible diffuse reflectance (UV-vis DRS) spectra (Model HVC-VUV). The white standard being used is spectralon. While treating the materials, spectra in the 200-800 nm region were recorded with a step size of 1 nm every 3 min. The Kubelka-Munk function (F(R)), derived from the recorded reflectance data, was used to present the results. On a chemisorption instrument (BELCAT II, Japan) with a thermal conductivity detector (TCD) for acidity information, methane temperature programmed desorption (CH₄-TPD) tests were performed for about 1 h before the CH₄ adsorption, about 100 mg of material was used charged into a quartz tube and heated with a He stream (ca. 20 mL min⁻¹). For 30 min, a 5% CH₄/He stream (ca. 20 mL min⁻¹) was expelled for adsorption after the temperature had dropped to 80° C. After purging with a He stream (20 mL min⁻¹) for 1 h, the physically adsorbed CH₄ was extracted. CH₄-TPD curves have been recorded at 10° C./min from 80 to 600° C. after the baseline was stabilized.

### Example 4: Catalytic Evolution

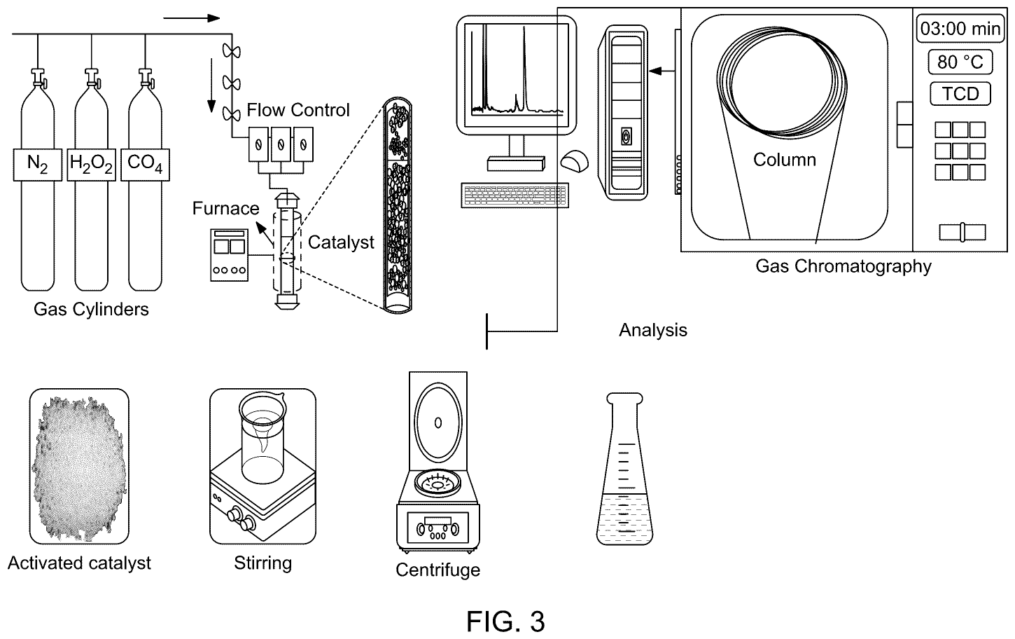

Catalyst powder pellets were sieved to 200-400 μm; about 0.70 g of catalyst was loaded in a fixed bed reactor, which was mounted vertically and coupled to an electric tube furnace. In a normal experiment, the material was heated at 2° C./min in the range of 100-500° C. in a 50 NmL/min O₂ flow and held constant for 8 h. The applied N₂ flow for 5 min removed different gas phase O₂ after cooling to 60° C. CH₄ and N₂ (5 and 30 NmL/min) were added to the reaction mixture. After 20 min, the catalyst was preheated using the same flow to 200° C. at a rate of 5° C./min. After 60 min, the methane-containing stream was terminated, and the catalyst bed was brought to room temperature. Methanol was extracted from the resulting substance by dispersing it in 5-40 m of water and stirring it vigorously for 2 h. In order to identify the liquid phase using gas chromatography (GC), centrifugation and filtration were utilized to separate the solid particles. FIG. 3 provides an illustration of the basic configuration of the reactor setup for direct methane oxidation to methanol.

### Example 5: Surface Morphology

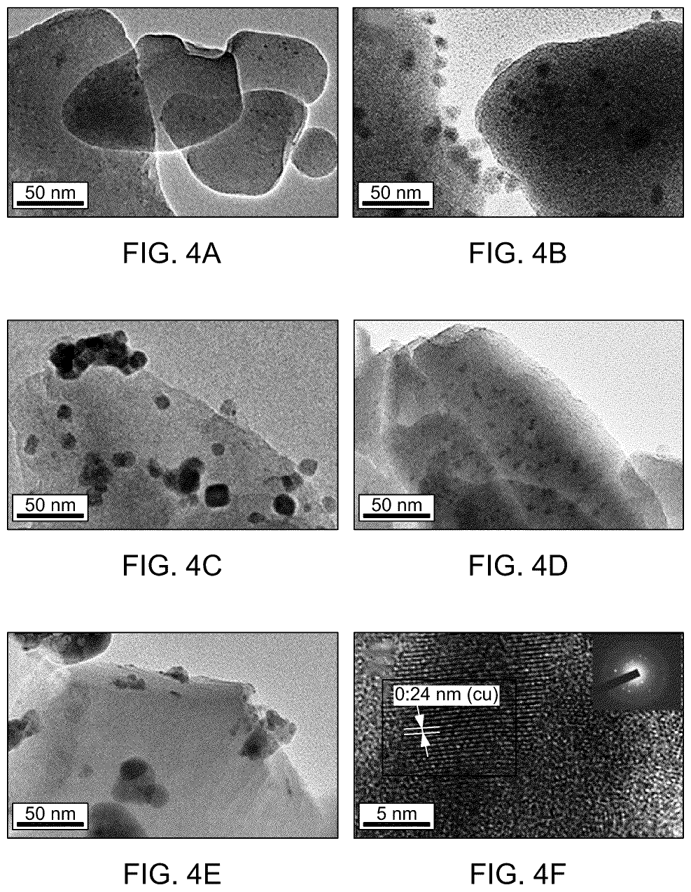

FIG. 4 is a representation of a number of synthesized catalysts that were produced using different methods. FIG. 4A depicts the TEM image of Cu exchanged MOR, demonstrating that the synthesis was carried out without any significant modifications. Notably, different metal particle sizes and dispersion were noticed using different preparation methods. These variations in metal particulate size and distribution result from the synthesis procedure. Among all catalysts, MOR (PM) illustrated large and agglomerate metal particles, as shown in FIG. 4E. These nanoparticles resulted from the depletion of CuO on the zeolite's exposed surface, the Cu-MOR (DS), shown in FIG. 4D includes the nanocrystals. The hydrophobic precursor introduced to the support during double solvent synthesis may have restricted the capillary action, allowing the dilute copper nitrate solution to permeate the support pores. CuO species interact strongly with the mordenite in this catalyst, presumably leading to the formation of nanocrystals within the pore network of zeolites (S. E. Bozbag, E. M. Alayon, J. Pech'a ̌cek, M. Nachtegaal, M. Ranocchiari, J. A. van Bokhoven, Methane to methanol over copper mordenite: yield improvement through multiple cycles and different synthesis techniques, Catal. Sci. Technol. 6 (13) (2016) 5011-5022, which is incorporated herein by reference in its entirety). In the case of MOR (IWI), prepared using the incipient wetness impregnation technique, agglomerated particles were also observed, as shown in FIG. 4C. Overall, the following is the sequence in which the average size of particles decreases: MOR (PM)>MOR (IWI)>MOR (WI)>MOR (DS). High-resolution TEM (HRTEM) micrograph of MOR (PM) is shown in FIG. 4F. It confirms the presence of Cu crystals as evidenced by the well-resolved (Cu: d=0.24 nm) crystalline lattice.

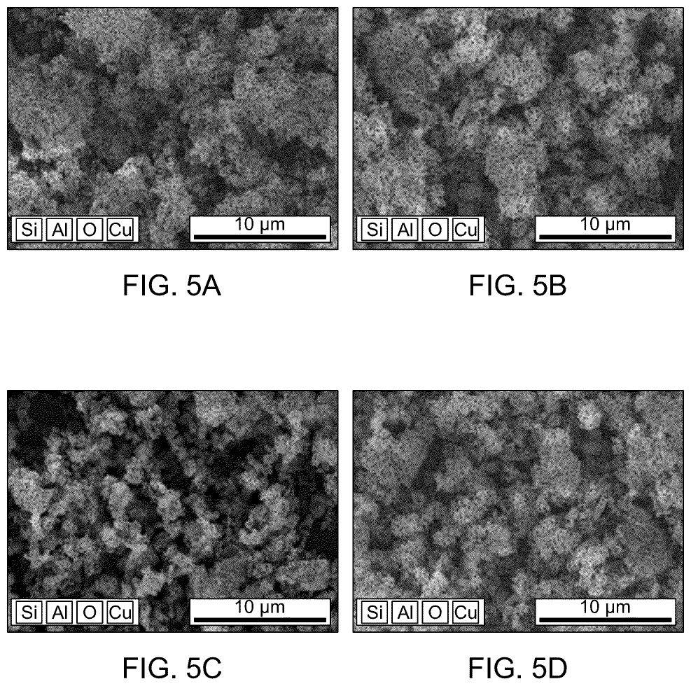

SEM/EDX mapping has been carried out to shed light on metal dispersion. SEM images of as-fabricated catalysts show CuO nanoparticle distribution and are revealed in FIG. 5A-5D. It is revealed that Cu incorporation did not destroy the zeolitic framework. However, the various metal loading techniques result in a small variation in morphological features. The elemental mapping images, are shown in FIGS. 5A-FIG. 5D, depict the CuO nanoparticles as bright pink dots (depicted as black dots). Wetness impregnation catalysts disperse and distribute CuO crystallites better than double solvent, incipient wet impregnation, and physical mixing techniques. The process of metal addition could be responsible for this behavior. The capillary action that causes the dilute copper nitrate solution to penetrate support pores may have been confined by the hydrophobic precursor introduced to the support during double solvent synthesis. Despite the ease and eco-friendly physical mixing technique, particle aggregation might have been observed after impregnation. Additionally, the increased surface area for reduction and reactivity results from the improved dispersion of CuO on Cu-MOR (WI). The simple and easily scaled wetness impregnation method proved significant in producing microscopic homogeneity that might prevent crystallite diffusion and improve catalytic performance.

N₂ adsorption-desorption study was used to elucidate the textural properties of all catalysts, and the results are shown in FIG. 6A and Table 1. All catalysts have minor isotherm differences, suggesting that the experimental conditions for different techniques did not change physicochemical characteristics. Notably, all catalysts exhibited reversible type IV isotherm, characteristic of microporous materials (G. Cheng, A. R. Hight Walker, Transmission electron microscopy characterization of colloidal copper nanoparticles and their chemical reactivity, Anal. Bioanal. Chem. 396 (2010) 1057-1069, which is incorporated herein by reference in its entirety). The microporosity of all the as-synthesized catalysts allowed N₂ adsorption to occur at reduced relative pressure (P/P₀), indicating intra- and interparticle pores at 0.45 and 0.93, respectively (L. Hu, X. Y. Wei, Y. H. Kang, X. H. Guo, M. L. Xu, Z. M. Zong, Mordenite-supported ruthenium catalyst for selective hydrodeoxygenation of lignin model compounds and lignin-derived bio-oil to produce cycloalkanes, J. Energy Inst. 96 (2021) 269-279, which is incorporated herein by reference in its entirety). In addition, the distribution of the pore sizes of all catalysts was measured using the Barrett-Joyner-Halenda (BJH) technique, shown in FIG. 7B. The surface areas of all as-synthesized catalysts decreased after processing in the following order: Cu-MOR (DS)>Cu-MOR (WI)>Cu-MOR (IWI)>Cu-MOR (PM). Cu metallic particles penetrating the pore spaces cause a decrease in surface area and porosity. The Cu-MOR (DS) and Cu-MOR (WI) catalysts prepared by wetness impregnation and double solvent methods significantly reduce surface area and pore volume. Thus, the simple and readily scalable wetness impregnation technique appears effective for generating microscopic homogeneity that might inhibit crystallite migration, causing improved catalytic activity.

TABLE 1
Textural Properties of the catalysts

| | Surface | Mesopore | Micropore | EDX | Average metal |
|---|---|---|---|---|---|
| | area ^{a} | volume ^{b} | volume ^{b} | analysis ^{c} | particle size ^{b} |
| Catalyst | (cm³/g) | (cm³/g) | (cm³/g) | (% wt.) | (nm) |
| Cu-MOR | 366 | 0.23 | 0.19 | 2.5 | — |
| Cu-MOR | 335 | 0.17 | 0.17 | 3.8 | 20 |
| (PM) | | | | | |
| Cu-MOR | 310 | 0.11 | 0.15 | 3.8 | 4 |
| (DS) | | | | | |
| Cu-MOR | 296 | 0.13 | 0.16 | 3.7 | 7 |
| (WI) | | | | | |
| Cu-MOR | 320 | 0.13 | 0.17 | 3.7 | 9 |
| (IWI) | | | | | |

^{a} refers to BET method.
^{b} refers to t-plot method.
^{c} refers to SEM-EDX.

The XRD patterns of the Cu-MOR (WI), Cu-MOR (IWI), Cu-MOR (DS), and Cu-MOR (PM) catalysts are shown in FIG. 7A. The diffraction patterns show the characteristic peaks that correspond to mordenite zeolite, among them are (200), (111), (310), (330), (150), (202), (350), (511), and (402) respectively (L. Hu, X. Y. Wei, Y. H. Kang, X. H. Guo, M. L. Xu, Z. M. Zong, Mordenite-supported ruthenium catalyst for selective hydrodeoxygenation of lignin model compounds and lignin-derived bio-oil to produce cycloalkanes, J. Energy Inst. 96 (2021) 269-279, which is incorporated herein by reference in its entirety). This confirms that no significant modification occurred during the preparations. The Cu metal was not identified as the crystallites are too small for the instrument to identify, and the low metallic load on the catalysts (2 wt. %).

In order to identify the characteristic functional groups associated with the as-synthesized catalysts, FTIR analysis was performed in a range of 4000-400 cm⁻¹ and presented in FIG. 7B. The external and internal asymmetric, external symmetric, and T-O bending vibrations were each allocated to absorption bands measured at 1220 cm⁻¹, 1085 cm⁻¹, 800 cm⁻¹, and 590 cm⁻¹, respectively (T. Kurniawan, O. Muraza, I. A. Bakare, M. A. Sanhoob, A. M. Al-Amer, Isomerization of n-butane over cost-effective mordenite catalysts fabricated via recrystallization of natural zeolites, Ind. Eng. Chem. Res. 57 (6) (2018) 1894-1902, which is incorporated herein by reference in its entirety). The double five-ring (D5R) structure, composed of tetrahedrally linked SiO₄ and AlO₄ units, is responsible for the vibration band in the mordenite framework measured at 590 cm⁻¹ (S. Narayanan, J. J. Vijaya, S. Sivasanker, M. Alam, P. Tamizhdurai, L. J. Kennedy, Characterization and catalytic reactivity of mordenite-investigation of selective oxidation of benzyl alcohol, Polyhedron 89 (2015) 289-296; and J. Ren, H. Li, Y. Jin, J. Zhu, S. Liu, J. Lin, Z. Li, Silica/titania composite-supported Ni catalysts for CO methanation: effects of Ti species on the activity, anti-sintering, and anti-coking properties, Appl. Catal., B 201 (2017) 561-572, each of which is incorporated herein by reference in their entireties). Si—OH bending vibrations were given a band at. 960 cm⁻¹, which led to more terminal silanol groups being found in Cu-MOR (WI) (N. Ghani, A. Jalil, S. Triwahyono, M. Aziz, A. Rahman, M. Hamid, S. Izan, M. Nawawi, et al., Tailored mesoporosity and acidity of shape-selective fibrous silica beta zeolite for enhanced toluene co-reaction with methanol, Chem. Eng. Sci. 193 (2019) 217-229, which is incorporated herein by reference in its entirety). This showed that Cu metal atoms interacted with SiO stretching vibrations near the siliceous substance structure. So, changes in the transmittance band could indicate the formation of Si—OH subgroup interfaces directed through Cu metal particles on MOR to create Si—O—Cu-bonds. Since the siliceous framework of the Cu-MOR (WI) catalyst was increased, CuO could disperse more on its surface using the wetness impregnation method. The significantly greater intensities were distinct from those of other catalysts. These results reveal the interaction of Cu metal particles, implying their firm connection and high dispersion across the Cu-MOR (WI). IR spectroscopy also reported and supported similar outcomes for integrating metallic ions into the zeolite framework (B. Tang, W. C. Song, S. Y. Li, E. C. Yang, X. J. Zhao, Post-synthesis of Zr-MOR as a robust solid acid catalyst for the ring-opening aminolysis of epoxides, Nouv. J. Chim. 42 (16) (2018) 13503-13511; and W. Dai, C. Wang, B. Tang, G. Wu, N. Guan, Z. Xie, M. Hunger, L. Li, Lewis's acid catalysis confined in zeolite cages as a strategy for sustainable heterogeneous hydration of epoxides, ACS Catal. 6 (5) (2016) 2955-2964, each of which is incorporated herein by reference in their entireties). These observations are highly consistent with EDS mapping and TEM results.

The copper species' coordination states and degree of agglomeration have also been determined using the DR UV-vis spectra (FIG. 8A). The O→Cu charge transition (CT) from lattice oxygen to localized Cu+/Cu²⁺ species fixed by the zeolite structure is responsible for the charge carrier band at 210-217 nm (S. R. Gomes, N. Bion, G. Blanchard, S. Rousseau, V. Belli'ere-Baca, V. Harl'e, D. Duprez, F. Epron, Thermodynamic and experimental studies of catalytic reforming of exhaust gas recirculation in gasoline engines, Appl. Catal. B Environ. 102 (1-2) (2011) 44-53; J. H. Park, H. J. Park, J. H. Baik, I. S. Nam, C. H. Shin, J. H. Lee, B. K. Cho, S. H. Oh, Hydrothermal stability of CuZSM5 catalyst in reducing NO by NH₃ for the urea selective catalytic reduction process, J. Catal. 240 (1) (2006) 47-57, each of which is incorporated herein by reference in their entireties). CuO species on the catalysts are responsible for the spectral absorption bands around 245 nm (L. Li, F. Zhang, N. Guan, M. Richter, R. Fricke, Selective catalytic reduction of NO by propane in excess oxygen over IrCu-ZSM-5 catalyst, Catal. Commun. 8 (3) (2007) 583-588, which is incorporated herein by reference in its entirety), and the band around 325 nm is evidence of the existence of oligomeric Cu²⁺— O₂—Cu²⁺ chains (I. Lezcano-Gonzalez, U. Deka, H. Van Der Bij, P. Paalanen, B. Arstad, B. A. M. Beale Weckhuysen, et al., Chemical deactivation of Cu-SSZ-13 ammonia selective catalytic reduction (NH₃-SCR) systems, Appl. Catal. B Environ. 154 (2014) 339-349, which is incorporated herein by reference in its entirety). The d→d transition of Cu²⁺ results in strong absorption between 500 and 800 nm, respectively (J. H. Park, H. J. Park, J. H. Baik, I. S. Nam, C. H. Shin, J. H. Lee, B. K. Cho, S. H. Oh, Hydrothermal stability of CuZSM5 catalyst in reducing NO by NH₃ for the urea selective catalytic reduction process, J. Catal. 240 (1) (2006) 47-57; and F. Bin, C. Song, G. Lv, J. Song, S. Wu, X. Li, Selective catalytic reduction of nitric oxide with ammonia over zirconium-doped copper/ZSM-5 catalysts, Appl. Catal. 150 (2014) 532-543, each of which is incorporated herein by reference in their entireties). The evaluation of the DR UV-Vis spectra reveals that copper species were ad ded to the Cu-MOR substances. Cu-MOR (WI) exhibited higher adsorption bands among all catalysts, showing that Cu-MOR (WI) owns high active sites and better metal dispersion.

### Example 6: CH₄ Temperature-Programmed Desorption (CH₄-TPD)

The effect of Cu loaded on the methane adsorption attraction of CH₄ with the catalysts was described. The CH₄ temperature-programmed desorption (CH₄-TPD) experiments were carried out, as illustrated in FIG. 8B. Increased desorption temperatures were observed for the catalyst prepared via the wetness impregnation technique, suggesting that the Cu addition could promote methane adsorption. Copper particles are responsible for adsorbing methane, dependent on metal dispersion and smaller particle sizes. This is highly consistent with TEM and EDS mapping results. Thus, the high dispersion and small particle size are expected to enhance the catalytic performance (F. Teng, W. Han, S. Liang, B. Gaugeu, R. Zong, Y. Zhu, Catalytic behavior of hydrothermally synthesized La0.5Sr0.5MnO₃ single-crystal cubes in the oxidation of CO and CH₄, J. Catal. 250 (1) (2007) 1-11; M. W. Kumthekar, U. S. Ozkan, Activity and temperature-programmed adsorption/desorption behavior of Pd@TiO₂ catalysts in NO/CH₄ reduction and NO decomposition reactions, Catal. Today 35 (1-2) (1997) 107-112, each of which is incorporated herein by reference in their entireties).

Ion exchange and hydrothermal techniques were used to synthesize the Cu-MOR, a potent and effective Cu-anchored based mordenite zeolite catalyst DMTM. Different methods for Cu incorporation on Cu-MOR via double solvent, physical mixing, wetness impregnation, and incipient wetness impregnation methods were employed. The Cu-MOR (WI) catalyst synthesized by wetness impregnation showed remarkable enhancement for catalytic performance, achieving outstanding results in methanol yield at 26.5 μmol g·cat⁻¹. The improved performance was linked to high metal dispersion and methane adsorption. This behavior is linked to Cu particle sizes and synthetic approaches, according to TEM and CH₄-TPD data. This shows that the wetness impregnation method has increased the number of active sites for absorbance and activation of methane molecules. Also, it was found that increasing the contact time of methane on the Cu-MOR (WI) favored the oxidation activity to produce methanol in the range of 10-60 min. During direct methanol production, a substantial influence on methanol yield can be achieved by increasing the activation temperature in the presence of oxygen. It is worth noting that increasing the extraction water volume from 5 mL to 20 mL per reaction batch resulted in a significant increase in extracted methanol. To acquire the considerable quantity of methanol that a catalytic cycle can produce, it is possible to conclude that the conditions of the extraction step should be selected with great care.

Catalytic performance for DMTM was evaluated using various catalysts synthesized using multiple techniques, as shown in FIG. 9A. At first, Cu-exchanged MOR depicted a methanol yield of 12 μmol gcat⁻¹, while adding Cu loading on the Cu-exchanged MOR further enhanced their catalytic performance. It is worth noting that the various techniques used for Cu incorporation, such as physical mixing, double solvent, physical mixing, incipient wetness impregnation, and wetness impregnation methods, significantly impact methanol production. It can be seen that the Cu-MOR (WI) catalyst prepared by the wetness impregnation method depicted higher performance (26.5 μmol gcat⁻¹) among all other catalysts. The order of their catalytic preference can be arranged in the order of Cu-MOR (WI)>Cu-MOR (IWI)>Cu-MOR (PM)>Cu-MOR (DS). These observations prove that Cu incorporation on Cu-exchanged MOR zeolite by wetness impregnation could produce more methanol during the DMTM. This showed that the wetness impregnation technique could facilitate the dispersion of Cu particles, leading to enhanced catalytic performance for the production of methanol. In other studies, methane oxidation was highly co-related with metal dispersion on the catalysts (J. Xie, R. Jin, A. Li, Y. Bi, Q. Ruan, Y. Deng, J. Tang, Highly selective oxidation of methane to methanol at ambient conditions by titanium dioxide-supported iron species, Nat. Catal. 1 (11) (2018) 889-896; Z. Liu, G. Xu, L. Zeng, W. Shi, Y. Wang, Y. Sun, H. He, Anchoring Pt-doped PdO nanoparticles on γ-Al₂O₃ with highly dispersed La sites to create a methane oxidation catalyst, Appl. Catal. 324 (2023), 122259; and Y. Lyu, J. N. Jocz, R. Xu, O. C. Williams, C. Sievers, Selective oxidation of methane to methanol over ceria-zirconia supported mono and bimetallic transition metal oxide catalysts, ChemCatChem 13 (12) (2021) 2832-2842). Cu-MOR (IWI) catalyst was chosen for further research to increase methanol production by interacting with methane over various time intervals, as depicted in FIG. 9B. This showed that the longer the contact time, the more methanol was formed on the Cu-MOR (WI). After adding methane, methanol reached 26.5 μmol gcat⁻¹ in 10 min at 200° C. In the initial 60 min of the reaction, methanol output increased steadily with time. After this reaction time, methane production steadily declined, resulting in a negligible yield increase within 10 min. It can be noticed that after 60 min, the methanol produced was 64.6 μmol gcat⁻¹. Activated catalysts had to react with methane for about 20 min to yield sufficient methyl alcohol (P. Tomkins, A. Mansouri, S. E. Bozbag, F. Krumeich, M. B. Park, E. M. Alayon, M. Ranocchiari, J. A. van Bokhoven, Isothermal cyclic conversion of methane into methanol over copper-exchanged zeolite at low temperature, Angew. Chem. 128 (18) (2016) 5557-5561; and M. J. Wulfers, S. Teketel, B. Ipek, R. F. Lobo, Conversion of methane to methanol on copper-containing small-pore zeolites and zeotypes, Chem comm 51 (21) (2015) 4447-4450). Activation temperature is crucial for catalytic performance for the DMTM (Y. Kim, T. Y. Kim, H. Lee, J. Yi, Distinct activation of Cu-MOR for direct oxidation of methane to methanol, Chem comm 53 (29) (2017) 4116-4119; and B. Han, Y. Yang, Y. Xu, U. Etim, K. Qiao, B. Xu, Z. Yan, A review of the direct oxidation of methane to methanol, Chin. J. Catal. 37 (8) (2016) 1206-1215). To investigate the impact on methanol generation, the Cu-MOR (IWI) catalyst was activated at various temperatures (100-500° C.), as depicted in FIG. 9C. It has been observed that raising the temperature at which the activation occurs led to increased methanol production. The highest output of methanol, 83.3 μmol gcat⁻¹, was obtained when the temperature was raised to 400° C. Increasing the temperature further reduced methanol output slightly, indicating that 400° C. is the preferred temperature for the Cu-MOR (IWI) catalyst to oxidize methane to methanol directly. The Cu species located at 8 MR windows of the side pockets could produce oxygen-bridged dicopper/tri copper sites by high-temperature treatment in O2, which were found to be reactive to methane (S. Grundner, M. A. Markovits, G. Li, M. Tromp, E. A. Pidko, E. J. Hensen, A. Jenty, M. Sanchez-Sanchez, J. A. Lercher, Single-site trinuclear copper oxygen clusters in mordenite for selective conversion of methane to methanol, Nature commun. 6 (1) (2015) 7546; P. Vanelderen, B. E. Snyder, M. L. Tsai, R. G. Hadt, J. Vancauwenbergh, O. Coussens, R. A. Schoonheydt, B. F. Sels, E. I. Solomon, Spectroscopic definition of the copper active sites in mordenite: selective methane oxidation, J. Am. Chem. Soc. 137 (19) (2015) 6383-6392; and A. R. Kulkarni, Z. J. Zhao, S. Siahrostami, J. K. Norskov, F. Studt, Monocopper active site for partial methane oxidation in Cu-exchanged 8MR zeolites, ACS Catal. 6 (10) (2016) 6531-6536). However, it was found that the activation step in oxygen has only a minor influence on methane-to-methanol performance. Compared to the results obtained, high-temperature (450° C.) catalyst activation decreased methanol yield (M. Ranocchiari, J. A. van Bokhoven, Isothermal cyclic conversion of methane into methanol over copper-exchanged zeolite at low temperature, Angew. Chem. 128 (18) (2016) 5557-5561). However, the findings of our study demonstrated that the catalytic activity of Cu-MOR (IWI) was noticeably increased when the activation temperature was raised to 400° C. Methanol yields of 37.3, 55.3, and 65.2 μmol gcat⁻¹ using the activation temperatures of 450° C., 550° C., and 650° C. were reported respectively. The last step in the catalytic cycle for methanol collection can be generally performed with ethanol or acetonitrile/water mixtures produced higher yields of methanol than extraction with dry acetonitrile or n-hexane in previous research. Thus, the solvent was considered to serve as a methanol-desorption reagent and a proton source for intermediary hydrolysis, possibly methoxy species (S. E. Bozbag, E. M. Alayon, J. Pech'a ̌cek, M. Nachtegaal, M. Ranocchiari, J. A. van Bokhoven, Methane to methanol over copper mordenite: yield improvement through multiple cycles and different synthesis techniques, Catal. Sci. Technol. 6 (13) (2016) 5011-5022; A. Sultana, T. Nanba, M. Sasaki, M. Haneda, K. Suzuki, H. Hamada, Selective catalytic reduction of NOx with NH₃ over different copper exchanged zeolites in the presence of decane, Catal. Today 164 (1) (2011) 495-499; and A. A. Reule, J. Shen, N. Semagina, Copper affects the location of zinc in bimetallic ion-exchanged mordenite, Chem Phys Chem 19 (12) (2018) 1500-1506, each of which is incorporated herein by reference in their entireties). Strong methanol adsorption in the offline extraction protocol on the zeolite surface can aggravate methanol extraction (R. Shah, J. D. Gale, M. C. Payne, Methanol adsorption in zeolites a first-principles study, J. Phys. Chem. 100 (28) (1996) 11688-11697). In the present disclosure, water was chosen as a solvent to produce methanol, and the impact of water amount on methanol production is shown in FIG. 9D. Increasing the extraction water volume from 5 mL to 20 mL per reaction batch (preferably, e.g., 0.7 g of calcined catalyst) resulted in an increase in extracted methanol. On the other hand, increasing the total amount of water used did not result in a perceptible increase in methanol after 20 mL. To acquire the greatest quantity of methanol that a catalytic cycle can produce, it is possible that the extraction step's conditions should be selected with great care.

Numerous modifications and variations of the present disclosure are possible in light of the above teachings. It is, therefore, to be understood that within the scope of the appended claims, the invention may be practiced otherwise than as specifically described herein.

1: A method for direct methane (CH₄) oxidation (DMTM) to methanol (CH₃OH), comprising:
introducing an oxygen-containing feed gas stream into a reactor containing a copper-loaded mordenite zeolite (Cu-MOR) catalyst comprising Cu-MOR catalyst particles having a porous structure, and an average particle size of 200 to 400 micrometers (μm);
wherein the Cu-MOR catalyst is at least one selected from the group consisting of a Cu-MOR (Cu-MOR-WI) catalyst made by wetness impregnation, and a Cu MOR (Cu-MOR-IWI) catalyst made by incipient wet impregnation;
passing the oxygen-containing feed gas stream through the reactor to contact the oxygen-containing feed gas stream with the Cu-MOR catalyst particles at a temperature of 100 to 500° C. to form an oxidized Cu-MOR catalyst;
terminating the introducing the oxygen-containing feed gas stream;
introducing and passing an CH₄-containing feed gas stream through the reactor to contact the CH₄-containing feed gas stream with the oxidized Cu-MOR catalyst at a temperature of 50 to 200° C. thereby converting at least a portion of the CH₄ to CH₃OH and regenerating the Cu-MOR catalyst particles to form a regenerated Cu-MOR catalyst, and producing a residue gas stream leaving the reactor;
wherein the CH₃OH is adsorbed on surfaces and pores of the regenerated Cu-MOR catalyst;
terminating the introducing the CH₄-containing feed gas stream and cooling the reactor; and
separating and collecting the CH₃OH.

2: The method of claim 1, wherein the CH₄ is present in the CH₄-containing feed gas stream at a concentration of 5 to 50 vol. % based on a total volume of the CH₄-containing feed gas stream.

3: The method of claim 1, wherein the CH₄-containing feed gas stream further comprises an inert gas selected from the group consisting of nitrogen, argon, and helium, and wherein a volume ratio of the CH₄ to the inert gas present in the CH₄-containing feed gas stream is in a range of 1:1 to 1:20.

4: The method of claim 3, wherein the CH₄-containing feed gas stream further comprises nitrogen, and wherein the residue gas stream leaving the reactor comprises methane, nitrogen, formaldehyde, carbon monoxide, carbon dioxide, and nitrogen oxides (NOₓ).

5: The method of claim 1, wherein the reactor is at least one selected from the group consisting of a fixed-bed reactor, a trickle-bed reactor, a moving bed reactor, a rotating bed reactor, a fluidized bed reactor, and a slurry reactor.

6: The method of claim 1, wherein the reactor is a fixed-bed reactor in the form of a cylindrical reactor comprising:
a top portion;
a cylindrical body portion;
a bottom portion;
a housing having an open top and open bottom supportably maintained with the cylindrical body portion;
wherein the Cu-MOR catalyst is supportably retained within the housing permitting fluid flow therethrough;
at least one propeller agitator is disposed in the bottom portion of the reactor;
wherein the bottom portion is cone shaped or pyramidal; and
wherein a plurality of recirculation tubes fluidly connects the bottom portion of the cylindrical reactor with the cylindrical body portion of the cylindrical reactor.

7: The method of claim 1, having a methanol production yield of about 10 to 30 μmol of methanol per gram of the Cu-MOR catalyst.

8: The method of claim 1, further comprising:
preparing the Cu-MOR-WI by:
mixing an aluminate salt and a silica material in an alkaline solution to form a first mixture;
heating the first mixture at a temperature of about 170° C. under pressure to form a crude product suspended in the first mixture;
removing the crude product from the first mixture, washing and calcining at a temperature of about 550° C. to form mordenite zeolite (MOR) having a porous structure;
mixing the MOR with a copper salt solution, and heating thereby depositing copper ions on surfaces and pores of the MOR;
calcining the MOR comprising the copper ions at a temperature of about 550° C. to form an ion exchange Cu-MOR (Cu-MOR-IE) catalyst;
mixing the Cu-MOR-IE catalyst and the copper salt solution to form a first modified Cu-MOR-IE catalyst in the form of particles suspended in the copper salt solution;
removing the first modified Cu-MOR-IE catalyst particles from the copper salt solution, and calcining at a temperature of about 550° C. to form the Cu-MOR-WI catalyst;
wherein the Cu-MOR-IE catalyst has a copper content of about 2.5 wt. % based on a total weight of the Cu-MOR-IE catalyst as determined by energy-dispersive X-ray spectroscopy (EDX); and
wherein the Cu-MOR-IE catalyst has a methanol production yield of about 12.4 μmol of methanol per gram of the Cu-MOR-IE catalyst.

9: The method of claim 8, wherein the aluminate salt is at least one selected from the group consisting of sodium aluminate and potassium aluminate.

10: The method of claim 8, wherein the copper salt solution comprises a copper salt selected from the group consisting of copper sulfate, copper nitrate, copper chloride, copper acetate, copper carbonate, copper phosphate, and/or a hydrate thereof.

11: The method of claim 8, wherein the Cu-MOR-WI catalyst comprises CuO nanoparticles having an average particle size of 7 nm.

12: The method of claim 8, wherein the Cu-MOR-WI catalyst has a copper content of about 3.7 wt. % based on a total weight of the Cu-MOR-WI catalyst as determined by EDX.

13: The method of claim 8, wherein the Cu-MOR-WI catalyst has a methanol production yield of about 26.5 μmol of methanol per gram of the Cu-MOR-WI catalyst.

14: The method of claim 8, wherein the Cu-MOR-WI catalyst comprises particles having a specific surface area in a range of 290 to 300 square meter per gram (m²/g).

15: The method of claim 8, wherein the Cu-MOR-WI catalyst comprises particles having a mesopore volume in a range of 0.1 to 0.15 cubic centimeter per gram (cm³/g), and a micropore volume in a range of 0.15 to 0.17 cm³/g.

16: The method of claim 8, further comprising:
preparing the Cu-MOR-IWI catalyst by:
applying the copper salt solution on surfaces and pores of the Cu-MOR-IE catalyst to form a second Cu-MOR-IE catalyst in the form of wetted particles; and
calcining the second Cu-MOR-IE catalyst at a temperature of about 550° C. to form the Cu-MOR-IWI catalyst comprising CuO nanoparticles having an average particle size of 9 nm.

17: The method of claim 16, wherein the Cu-MOR-IWI catalyst has a copper content of about 3.7 wt. % based on a total weight of the Cu-MOR-IWI catalyst as determined by EDX.

18: The method of claim 16, wherein the Cu-MOR-IWI catalyst has a methanol production yield of about 23.2 μmol of methanol per gram of the Cu-MOR-IWI catalyst.

19: The method of claim 16, wherein the Cu-MOR-IWI catalyst comprises particles having a specific surface area in a range of 310 to 330 m²/g.

20: The method of claim 16, the Cu-MOR-IWI catalyst comprises particles having a mesopore volume in a range of 0.1 to 0.15 cm³/g, and a micropore volume in a range of 0.16 to 0.18 cm³/g.
